(19) United States
(12) Patent Application Publication
(10) Pub. No.: US 2024/0350581 A1
(43) Pub. Date: Oct. 24, 2024

(54) METHODS FOR TREATMENT OF NEURON DEGENERATION

(71) Applicant: THE UNIVERSITY OF WESTERN ONTARIO, London (CA)

(72) Inventors: Michael STRONG, London (CA); Cristian DROPPELMANN, London (CA); Kathryn VOLKENING, West Lorne (CA); Danae CAMPOS-MELO, London (CA); Hind AMZIL, Toronto (CA)

(73) Assignee: THE UNIVERSITY OF WESTERN ONTARIO, London, ON (CA)

(21) Appl. No.: 18/288,813

(22) PCT Filed: Apr. 29, 2022
(86) PCT No.: PCT/CA2022/050665

§ 371 Date: Oct. 29, 2023

Related U.S. Application Data
(60) Provisional application No. 63/181,452, filed on Apr. 29, 2021.

(51) Int. Cl. A61K 38/17; A61P 25/28; C12N 15/113

(52) U.S. Cl. CPC A61K 38/1709; A61P 25/28; C12N 15/113; C12N 2310/14; C12N 2320/31

(57) ABSTRACT

A method for reducing, inhibiting, preventing or treating cytotoxicity in a cell, the method comprising expressing or overexpressing in the cell a Rho guanine nucleotide exchange factor (RGNEF), or a RGNEF analog or agonist, or a leucine rich domain of RGNEF, whereby the cytotoxicity in the cell is reduced, inhibited, prevented or treated. Also a method of shifting the survival curve of a subject and thereby increasing life expectancy of the subject by administering to the subject a physiologically effective amount of a peptide comprising A Rho Guanine Nucleotide Exchange Factor (RGNEF) protein, or a RGNEF analog or agonist, or a leucine rich domain of RGNEF, or expressing or overexpressing RGNEF or a RGNEF analog or agonist or a leucine rich domain of RGNEF in the subject.

### FIELD OF THE INVENTION

The present invention relates generally to the field of use of TDP-43 antagonists in the treatment of TDP-43 associated diseases and in the treatment of neurodegenerative diseases.

### BACKGROUND

Rho Guanine Nucleotide Exchange Factor (RGNEF) is a 190 kDa protein that is unique in the human proteome for its ability to act both as a guanine exchange factor (GEF) and an RNA-binding protein which regulates mRNA stability (Droppelmann et al., 2014; Droppelmann et al., 2013; Volkening et al., 2010). Under normal physiological conditions, RGNEF is mainly cytoplasmic and localizes at moderate levels to the nucleus (Droppelmann et al., 2013). RGNEF and its murine homologue p190RhoGEF serve as survival factors in response to oxidative and osmotic stress in vitro (Cheung et al., 2017; Wu et al., 2003) and as a stress response factor in vivo, showing significantly upregulated levels following distal sciatic nerve injury in mice (Cheung et al., 2017). RGNEF has been demonstrated to have a relevant role in ALS forming cytoplasmic inclusions that co-aggregate with other ALS-related proteins in spinal cord motor neurons (Droppelmann et al., 2013; Keller et al., 2012). Additionally, it has been observed that RGNEF plays a role in colon carcinoma through its interaction with focal adhesion kinase (FAK) which facilitates tumor growth and invasion (Yu et al., 2011).

RGNEF has several functional domains, including a Leucine-rich region, a cysteine-rich Zn binding domain, a Dbl homology domain (DH), a Pleckstrin homology domain (PH) and an RNA-binding domain (Tavolieri et al., 2019; see FIG. 1A).

### ALS

Amyotrophic Lateral Sclerosis or ALS, also known as Lou Gehrig's disease, remains one of the most devastating diseases of our time. Affecting individuals in the prime of their lives, the disease process underlying ALS leads to a profound, progressive loss of all muscle bulk and control, culminating in death from respiratory failure within 3 to 5 years of the first symptom for the vast majority of patients. To date, there are no therapies which can arrest its progression. In part, this is because ALS is not one disease, but rather a syndrome that can be triggered through a range of different genetic factors, or spontaneously occur. However, common to each of these is the fact that individual motor neurons, those which are responsible for driving the function of muscle, degenerates. Under the microscope, this seemingly diverse origin of the disease melts away as the hallmark of all variants of ALS is the presence of unique protein inclusions, the most common of which are composed of RNA binding proteins (RNA-BPs).

Amongst all of the RNA-BPs now known to be abnormally processed in ALS, one stands out: the Tar DNA binding protein of 43 kilodalton (TDP-43). In virtually every form of ALS, the neuropathological signature of the disease process is not only the marked upregulation of expression of TDP-43 within motor neurons, a process accompanied by the movement of TDP-43 from a predominantly nuclear localization to a predominantly cytosolic localization, but also the formation of neuronal cytoplasmic inclusions (NCIs) (see FIG. 2).

### SUMMARY

In one embodiment, the present disclosure provides a method for reducing, inhibiting, preventing or treating cytotoxicity in a cell, the method comprising expressing or overexpressing in the cell a Rho guanine nucleotide exchange factor (RGNEF), or a RGNEF analog or agonist, or a leucine rich domain of the RGNEF, whereby the cytotoxicity in the cell is reduced, inhibited, prevented or treated. In one aspect, the cytotoxicity is TDP-43 induced. In another aspect the cell is a neuron.

In another embodiment, the present disclosure is a use of a Rho guanine nucleotide exchange factor (RGNEF), a RGNEF analog or agonist, or a leucine rich domain of the RGNEF for reducing, inhibiting, preventing or treating cytotoxicity in a cell. In one aspect, the cytotoxicity is TDP-43 induced.

In another embodiment, the present invention is a method of shifting the survival curve of a subject and thereby increasing life expectancy of the subject, comprising administering to the subject a physiologically effective amount of a peptide comprising a Rho guanine nucleotide exchange factor (RGNEF), a RGNEF analog or agonist, or a leucine rich domain of the RGNEF, or expressing or overexpressing RGNEF, a RGNEF analog or agonist, or a leucine rich domain of RGNEF in the subject.

In another embodiment, the present disclosure relates to a use of a peptide comprising a Rho guanine nucleotide exchange factor (RGNEF), or a RGNEF analog or agonist, or a peptide comprising a leucine rich domain of RGNEF, or expressing or overexpressing RGNEF, a RGNEF analog or agonist or a leucine rich domain of the RGNEF for shifting the survival curve of a subject and thereby increasing life expectancy of the subject.

In another embodiment, the present invention relates to a method of treating a condition associated with TDP-43 cytotoxicity or a neurodegenerative condition in a subject, the method comprising administering to the subject a TDP-43 antagonist.

In one embodiment of the method of treating a condition associated with TDP-43 cytotoxicity or a neurodegenerative condition in a subject, the TDP-43 antagonist comprises a Rho guanine nucleotide exchange factor (RGNEF) or a RGNEF agonist or analog.

In another embodiment of the method of treating a condition associated with TDP-43 cytotoxicity or a neurodegenerative condition in a subject, the TDP-43 antagonist comprises a leucine-rich RGNEF domain.

In another embodiment of the method of treating a condition associated with TDP-43 cytotoxicity or a neurodegenerative condition in a subject, the TDP-43 antagonist is a DNA molecule that encodes for RGNEF, for a RGNEF agonist, or for a RGNEF leucine-rich domain.

In another embodiment of the method of treating a condition associated with TDP-43 cytotoxicity or a neurodegenerative condition in a subject, the TDP-associated disorder or the neurodegenerative condition is Amyotrophic Lateral Sclerosis.

In another embodiment, the present disclosure provides for a composition for treating a condition associated with TDP-43 cytotoxicity or a neurodegenerative condition, the composition comprising a TDP-43 antagonist, and a pharmaceutically acceptable carrier.

In one embodiment of the composition for treating a condition associated with TDP-43 cytotoxicity or a neurodegenerative condition, the TDP-43 antagonist comprises a Rho guanine nucleotide exchange factor (RGNEF) or a RGNEF agonist.

In another embodiment of the composition for treating a condition associated with TDP-43 cytotoxicity or a neurodegenerative condition, the TDP-43 antagonist comprises a leucine-rich RGNEF domain.

In another embodiment of the composition for treating a condition associated with TDP-43 cytotoxicity or a neurodegenerative condition, the TDP-43 antagonist is a DNA molecule that encodes for RGNEF, for a RGNEF agonist, or for a RGNEF leucine-rich domain.

In another embodiment of the composition for treating a condition associated with TDP-43 cytotoxicity or a neurodegenerative condition, the TDP-associated disorder or the neurodegenerative condition is Amyotrophic Lateral Sclerosis.

In another embodiment, the present disclosure provides a use of a TDP-43 antagonist for the treatment of a condition associated with TDP cytotoxicity or for the treatment of a neurodegenerative condition.

In one embodiment of the use of a TDP-43 antagonist for the treatment of a condition associated with TDP cytotoxicity or for the treatment of a neurodegenerative condition, the TDP-43 antagonist comprises a Rho guanine nucleotide exchange factor (RGNEF) or a RGNEF agonist.

In another embodiment of the use of a TDP-43 antagonist for the treatment of a condition associated with TDP cytotoxicity or for the treatment of a neurodegenerative condition, the TDP-43 antagonist comprises a leucine-rich RGNEF domain.

In another embodiment of the use of a TDP-43 antagonist for the treatment of a condition associated with TDP cytotoxicity or for the treatment of a neurodegenerative condition, the TDP-43 antagonist is a DNA molecule that encodes for RGNEF, or for a RGNEF agonist, or for a leucine-rich domain of RGNEF.

In another embodiment of the use of a TDP-43 antagonist for the treatment of a condition associated with TDP cytotoxicity or for the treatment of a neurodegenerative condition, the TDP-associated condition is Amyotrophic Lateral Sclerosis.

In another embodiment, the present disclosure relates to a method of translocating a protein of interest to the nucleous of a cell, the method comprising fusing the protein of interest to a Pleckstrin homology domain (PH) domain of a Rho Guanine Nucleotide Exchange Factor (RGNEF).

In another embodiment, the present disclosure relates to a use of a Pleckstrin homology domain (PH) domain of a Rho Guanine Nucleotide Exchange Factor (RGNEF) to translocate a protein to the nucleous of a cell.

### BRIEF DESCRIPTION OF THE FIGURES

The invention will be better understood and objects of the invention will become apparent when consideration is given to the following detailed description thereof. Such description makes reference to the annexed drawings wherein:

FIGS. 1A to 1C. FIG. 1A is a schematic of RGNEF structure. Structurally, RGNEF has five important domains: a leucine-rich region and a cysteine-rich Zn-binding domain in the amino terminal half of the protein; a Dbl homology domain (DH), a Pleckstrin homology domain (PH) and a RNA-binding domain in the carboxyl half of the protein. FIG. 1B is the amino acid sequence of the PH domain of RGNEF. The nuclear localization signal (NLS) is shown in red with the basic residues marked with asterisks. Grey, orange, and blue underline show the structural prediction and represent coil, β-Strand, and α-Helix, respectively. FIG. 1C: Ribbon and spacefill models of the PH domain of RGNEF. The NLS is shown in red with the basic residues colored in yellow.

FIGS. 2A to 2C—RGNEF co-aggregates with TDP-43 in ALS motor neurons. 2A: anti-RGNEF, 2B: anti-TDP-43 (red), 2C merge of 2A and 2B. 3D reconstruction after Z-stack analysis of immunofluorescence showing RGNEF (green) and TDP-43 (red) cytoplasmic inclusions in a spinal cord motor neuron of an ALS patient.

FIGS. 3A and 3B—RGNEF increases survival of cells under stress conditions. HEK293T cells were exposed to either an oxidative (sodium arsenite) or osmotic (sorbitol) stress and then cell survival assayed using either a MTT assay (3A) or commercial cytotoxicity assay (3B, basal conditions). In both instances, RGNEF conferred a significant inhibition of the toxicity and enhanced cell survival (Cheung et al., 2017).

FIGS. 4A and 4B—Deletion of the leucine-rich region of RGNEF significantly modifies its RNA destabilizing activity. 4A: RGNEF constructs used in examining NEFL mRNA stability (L=leucine-rich region; Zn=zinc binding domain; GEF=Dbl homology domain domain; PH=pleckstrin homology domain; RBD=RNA binding domain). 4B: Luciferase assay showing the effect of the different RGNEF constructs of FIG. 4A over the stability of firefly luciferase mRNA bound to NEFL 3′UTR as previously described (Droppelmann et al., 2013). Negative values imply mRNA destabilization and positive values imply mRNA stabilization (*=p<0.05). Note the dramatic loss of NEFL mRNA stability when RGNEF is expressed with a N-terminal deletion (bars 3, 4, and 5).

FIGS. 5A and 5B—Endogenous RGNEF colocalizes with endogenous TDP-43 immunoreactive intracellular aggregates following metabolic stress. HEK293T cells subjected to a lactate overdose stress form intracytoplasmic inclusions immunoreactive to RGNEF (lower panel FIG. 5A) in HEK293T cells expressing only the leucine rich domain (flag tagged) (upper pane FIG. 5A). Final panel in each represents correlation intensity analysis in which only the top co-localized pixels are evident, demonstrated the clear incorporation of RGNEF into TDP-43 intracellular aggregates in response to cell stress.

FIG. 5B demonstrates the colocalization of TDP-43 immunoreactive neuronal cytoplasmic inclusions in which full length RGNEF and TDP-43 are colocalized.

FIGS. 6A and 6B—The leucine rich domain of RGNEF and full length RGNEF participate in a complex containing TDP-43. HEK293T cells were transfected with flag-tagged leucine rich domain of RGNEF and myc-tagged TDP-43 and then immunoprecipitations performed following cross-linking with DTSSP (FIG. 6A). In the left hand panel of FIG. 6A, the immunoprecipitation was performed using anti-myc antibodies and the western blot developed using an anti-flag antibody; in this case TDP-43 immunoprecipitates leucine rich domain of RGNEF. In the right hand panel, the blot confirms the immunoprecipitation of myc tagged TDP-43 (Droppelmann et al., unpublished observations). In FIG. 6B, cell lysates of HEK cells expressing myc-tagged full length RGNEF were immunoprecipitated and then western blot using anti-myc antibodies was performed. Lane 2 demonstrates the immunoprecipitation of the myc-tagged RGNEF. In lane 3 (red box), samples were immunprecipitated using a rabbit polyclonal antibody to human TDP-43 and then the Western blot immunoreacted with the anti-myc antibody. In this instance, anti-TDP-43 antibody immunoprecipitated myc-tagged RGNEF. Of note, this was also demonstrated for FUS/TLS immunoprecipitation of myc-tagged RGNEF confirmed the key role for RGNEF in concert with the two major RNA binding proteins associated with ALS (TDP-43 and FUS/TLS, respectively) (lane 4). The remaining lanes represent appropriate controls (Keller et al., 2012).

FIG. 7—RGNEF regulates the stability of TDP-43 through its 3′UTR and induces a reduction in TDP-43 protein levels.

FIG. 8—RGNEF participates in miRNA biogenesis, including the regulation of miRNAs critical to the degradation of TDP-43 mRNA (Hawley et al., unpublished observations). Endogenous RGNEF expression was suppressed in HEK293T cells using siRGNEF (upper left panel; control using scramble siRNA). Using a microarray analysis for miRNA expression, we observed both significant down-regulation of a pool of miRNAs but also upregulation. Amongst these were miRNAs integral to the regulation of TDP-43 expression. We confirmed that RGNEF co-immunoprecipated with components of miRNA biogenesis (bottom left panel). Further studies are ongoing to explore the mechanism(s) of this key role of RGNEF in miRNA biogenesis.

FIG. 9A The expression of full length RGNEF reduces wtTDP-43 toxicity in HEK293T cells. HEK 293T cells were transiently transfected with full length RGNEF alone (column 2), wt type TDP-43 (column 3) or both RGNEF and wtTDP-43 (column 4). The CytoTox 96® Non-Radioactive Cytotoxicity Assay kit (Promega) was used for the measurement of cytotoxicity 48 h post-transfection. An empty vector was transfected as control (column 1). RGNEF alone reduces cell death under basal conditions while TDP-43 expression leads to an increased cell death. The co-expression of RGNEF reduces the toxic effect of wt TDP-43 expression.

FIG. 9B—RGNEF decreases the cytotoxicity induced by TDP-43A315T in vitro. A cytotoxicity assay was performed in HEK293T cells transfected with vectors expressing RGNEF alone (column 2), mutant TDP-43 associated with familial ALS (TDP-43A315T) and both RGNEF and TDP-43A315T. An empty vector was transfected as control (column 1). The CytoTox 96® Non-Radioactive Cytotoxicity Assay kit (Promega) was used for the measurement of cytotoxicity 48 h post-transfection. In the presence of RGNEF alone, a reduced cytotoxicity consistent with our survival experiments using MTT is observed. When TDP-43A315T is expressed alone, a significant increase in cytotoxicity is observed as expected. Interestingly, when RGNEF is co-transfected with TDP-43 wt (FIG. 9A) or TDP-43A315T (FIG. 9), a significant decrease of the cytotoxicity of either variant of TDP-43 is observed (***=p<0.001). Also, we observed a reduction of cytotoxicity only in presence of RGNEF consistent with our previous report.

FIG. 10—RGNEF expression extends the lifespan offruit flies. Lifespan measurement of fruit flies expressing RGNEF in the nervous system (Elav-gal4/UAS-RGNEF; pan-neuronal expression) compared with control flies carrying the transgene (RGNEF control) but not expressing the protein and flies expressing the driver alone (elav-w-control). Flies expressing RGNEF show an extended lifespan compared with the non-expressing ones. In contrast (right hand panel) the expression of wild type TDP-43 led to a significant reduction in survival (two separate lines demonstrated).

FIGS. 11A and 11B The toxicity ofwild-type TDP-43 is abolished or significantly reduced in the presence of either full length RGNEF or the leucine rich domain of RGNEF alone. Double transgenic flies were created expressing wild-type TDP-43 accompanied by constructs expressing either full length RGNEF (RGNEF) or the leucine rich domain of RGNEF (LeuR) with the latter two under the expression driven by either a pan-neuronal driver (Elav) or a motor neuron specific driver (D42). Regardless of the driver or the construct, the toxicity of TDP-43 was significantly reduced or fully inhibited as measured by either negative geotaxis (climbing assay; FIG. 11A) or survival (FIG. 11B). We also demonstrated that the expression of TDP-43 was comparable amongst all fly lines (data not shown).

FIG. 12. Both the full-length RGNEF and the Leucine-rich domain dramatically abolish the toxic phenotype induced by TDP-43 in fruit flies. Motor function measurement at day 5 of adult stage (climbing scores, where 4 is normal motor function and 1 is no movement) of fruit flies expressing TDP-43 (Elav-gal4/UAS-TDP-43), RGNEF and TDP-43 (Elav-gal4/UAS-TDP-43/UAS-RGNEF), and RGNEF-L-rich region and TDP-43 (Elav-gal4/UAS-TDP-43/UAS-L-rich) in the nervous system compared with control flies carrying the transgene (UAS-RGNEF) but not expressing the protein. Notably, when either the RGNEF or the Leucine-rich domain are expressed, the motor deficit induced by TDP-43 is completely abolished.

FIG. 13 Both RGNEF and the leucine rich domain of RGNEF strongly reduces the toxicity of TDP-43 in the eye of drosophila. Photographs of Drosophila eyes from single transgenic flies expressing RGNEF (GMR-RGNEF) or LeuR (GMR-LeuR) and double transgenic flies co-expressing RGNEF and TDP-43 (GMR-RGNEF;TDP-43) or LeuR and TDP-43 (GMR-LeuR;TDP-43) using the eye specific driver GMR. The yellow arrows (two arrows on the left side of the photograph at approximately 0800 and 1000 hrs) in the GMR-TDP-43 flies indicates discoloration and lose of ommatidia architecture. The blue arrow (single arrow on the right side of the photograph located at approximately 0230 hours) indicates presence of necrotic tissue. GMR-w is used as control.

FIGS. 14A and 14B. Co-localization of the leucine rich domain ofRGNEF and TDP-43 in neurons within the eye and brain of double transgenic drosophila. FIG. 14A: Microphotographic images illustrating the co-localization within neurons of the eye of flagged-leucine rich domain of RGNEF (f-leuR) with TDP-43. 14B: Microphotographic images illustrating the co-localization of f-LeuR with TDP-43 within brain neurons subserving sight. Similar to the pathology observed in both human spinal motor neurons and in vitro using HEK293T cells under stress, intraneuronal aggregates of TDP-43 are observed in double transgenic flies expressing wild type TDP-43 and again similar to the previous observations, co-localization with the leucine rich domain of RGNEF is observed.

FIGS. 15A-15E. The NLS in the PH domain of RGNEF is essential for its nuclear localization. (15A) Schematic representation of the three RGNEF constructs used in subcellular localization experiments of Example 2. (15B) Representative confocal images of HEK293T cells transfected with myc-tagged RGNEF-wt or the mutant constructs, which both lacking a functional NLS. White indicates co-localized pixels. Scale bar=15 μm. (15C) Quantification of cellular localization of confocal images in 16B. Both RGNEF-APH and RGNEF-mutNLS showed significantly lower levels of cells showing nuclear localization than RGNEF-wt. Means were compared by ANOVA using Tukey's post-hoc test and p values are indicated. Graph shows means±SEM. (15D) Western blot performed using HEK293T cells lysates transfected with myc-tagged RGNEF constructs and separated by subcellular fractionation. α-Lamin A/C (exclusively nuclear) and α-GAPDH are used as loading controls. (15E) Densitometry analysis of experiment showed in D. Nuclear fractions from cells transfected with either mutant RGNEF construct showed lower levels of protein than those expressing RGNEF-wt. Means compared by ANOVA using Tukey's post-hoc test and p values are indicated.

FIG. 16. RGNEF mutants lacking the NLS show lower levels of nuclear localization in a neuron-like cell type. Representative confocal images of differentiated SH-SY5Y cells transfected with myc-tagged RGNEF-wt or RGNEF-mutNLS. Cells were differentiating using 10 μM retinoic acid. β-Tubulin III was used as a marker of neuronlike phenotype to distinguish differentiated from non-differentiated cells. Moderate levels of nuclear localization were observed in cells transfected with RGNEF-wt, whereas cells transfected with either RGNEF-mutNLS do not. Scale bar=15 μm Graph shows means±SEM.

FIGS. 17A to 17C. Wild type, but not the mutated PH domain, is able to translocate a 160 kDa fusion protein to the nucleus. (17A) Schematic representation of the pHM830 constructs used in this experiment. (17B) Representative confocal images of HEK293 T cells transfected with empty pHM830 vector, pHM830 expressing the wild-type PH domain (830-PH-wt) or expressing the PH domain containing NLS mutations (830-PH-mutNLS). Scale bar=15 μm. (17C) Quantification of cellular localization of confocal images of the experiment showed in FIG. 17B. Cells transfected with pH830-PH-wt show a significantly higher percentage of nuclear localization compared to these transfected with either empty pHM830 vector or pH830-PHmNLS. Means were compared by ANOVA using Tukey's post-hoc test and p values are indicated. Graph shows means±SEM.

FIGS. 18A to 18C. The PH domain of RGNEF contains an active NES. (18A) Schematic representation of the pH840 constructs used in this experiment. Note the presence of the SV40 NLS C-terminal to the PH domain insert. (18B) Representative confocal images of HEK293T cells transfected with empty pHM830 and pHM840 vectors, pHM840-PH-wt, and pHM840-PH-mutNLS. Scale bar=15 μm. (18C) Quantification of nuclear: cytoplasmic ratios generated from confocal images of HEK293T transfected with constructs showed in FIG. 18A. Cells transfected with empty pHM840 vector showed very high levels of nuclear localization, as expected given the NLS present in the vector. Cells transfected with the wild-type PH domain (840-PH-wt) showed lower levels of nuclear localization and those transfected with the mutated NLS PH domain (840-mutNLS) showed even lower levels. Means compared by ANOVA using Tukey's post-hoc test. Graph shows means+SEM.

FIGS. 19A to 19B. The nuclear export of fusion proteins containing the PH domain of RGNEF depends of exportin-1. (19A) Representative confocal images of HEK293T cells expressing 840-PH-wt and 840-PH-mutNLS and treated with either Leptomycin-B (LMB) or ethanol (vehicle). Scale bar=15 μm. (19B) nuclear:cytoplasmic ratios generated for HEK293T cells transfected with either of the two pHM840 constructs and treated with either 20 nM LMB or equal volume of ethanol (vehicle). Means were compared by ANOVA using Tukey's post-hoc test. Graph shows means±SEM.

FIG. 20. Depletion of RGNEF from SH-SY5Y neuronal cells alters significantly the expression of 1607 genes. Total RNA was extracted from RNA interference negative control and RGNEF depleted SH-SY5Y cells and RNAseq was performed comparing depleted versus control cells. The figure shows a schematic representation of RNA-seq data obtained from the experiment. Up- and downregulated genes are reported as red and green dots, respectively. Not differentially expressed genes (DEGs) are represented as grey dots. The horizontal dashed line indicates the statistical significance threshold (padjust=0.05); 319 genes are up-regulated and 1288 genes are down-regulated.

FIG. 21. KEGG pathway analysis of RGNEF depletion RNA-seq data. The KEGG pathway analysis of the RNAseq data of RGNEF-depleted SH-SY5Y cells shows that the expression of several genes related to axon guidance is altered after RGNEF depletion.

### DETAILED DESCRIPTION

Throughout this application, the text refers to various embodiments of the present compositions and methods. The various embodiments described are meant to provide a variety of illustrative examples and should not be construed as descriptions of alternative species. Rather it should be noted that the descriptions of various embodiments provided herein may be of overlapping scope. The embodiments discussed herein are merely illustrative and are not meant to limit the scope of the present invention.

Also throughout this disclosure, various publications, patents and published patent specifications are referenced by an identifying citation or an Arabic number, the complete bibliographic citation for which is found immediately preceding the claims. The disclosures of these publications, patents and published patent specifications are hereby incorporated by reference into the present disclosure in their entirety to more fully describe the state of the art to which this invention pertains.

### Definitions

As used in the specification and claims, the singular form “a,” “an” and “the” include plural references unless the context clearly dictates otherwise. For example, the term “a cell” includes a plurality of cells, including mixtures thereof.

As used herein, the term “comprising” is intended to mean that the compositions and methods include the recited elements, but not excluding others. “Consisting essentially of” when used to define compositions and methods, shall mean excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients. Embodiments defined by each of these transition terms are within the scope of this invention.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into subranges as discussed above. All numerical designations, e.g., pH, temperature, time, concentration, and molecular weight, including ranges, are approximations which are varied (+) or (−) by increments of 0.1 or 1.0 as is appropriate. It is to be understood, although not always explicitly stated that all numerical designations are preceded by the term “about” which includes a standard deviation of about 15%, or alternatively about 10% or alternatively about 5%. It also is to be understood, although not always explicitly stated, that the reagents described herein are merely exemplary and that equivalents of such are known in the art.

“Antagonist” refers to a molecule or substance that interferes directly or indirectly with or inhibits the physiological action of another molecule or substance.

As used herein, an “antibody” includes whole antibodies and any antigen binding fragment or a single chain thereof. Thus the term “antibody” includes any protein or peptide containing molecule that comprises at least a portion of an immunoglobulin molecule. Examples of such include, but are not limited to a complementarity determining region (CDR) of a heavy or light chain or a ligand binding portion thereof, a heavy chain or light chain variable region, a heavy chain or light chain constant region, a framework (FR) region or any portion thereof or at least one portion of a binding protein.

The antibodies can be polyclonal or monoclonal and can be isolated from any suitable biological source, e.g., murine, rat, sheep or canine.

An “effective amount” is an amount sufficient to effect beneficial or desired results. An effective amount can be administered in one or more administrations, applications or dosages. Such delivery is dependent on a number of variables including the time period for which the individual dosage unit is to be used, the bioavailability of the therapeutic agent, the route of administration, etc. It is understood, however, that specific dose levels of the therapeutic agents of the present invention for any particular subject depends upon a variety of factors including the activity of the specific compound employed, bioavailability of the compound, the route of administration, the age of the animal and its body weight, general health, sex, the diet of the animal, the time of administration, the rate of excretion, the drug combination, and the severity of the particular disorder being treated and form of administration. Treatment dosages generally may be titrated to optimize safety and efficacy. Typically, dosage-effect relationships from in vitro and/or in vivo tests initially can provide useful guidance on the proper doses for patient administration. Studies in animal models generally may be used for guidance regarding effective dosages for treatment of diseases. In general, one will desire to administer an amount of the compound that is effective to achieve a serum level commensurate with the concentrations found to be effective in vitro. Thus, where a compound is found to demonstrate in vitro activity, for example as noted in the Tables discussed below one can extrapolate to an effective dosage for administration in vivo. These considerations, as well as effective formulations and administration procedures are well known in the art and are described in standard textbooks. Consistent with this definition and as used herein, the term “therapeutically effective amount” is an amount sufficient to treat a specified disorder or disease or alternatively to obtain a pharmacological response treating a glioblastoma.

As used herein, “treating” or “treatment” of a disease in a patient refers to (1) preventing the symptoms or disease from occurring in an animal that is predisposed or does not yet display symptoms of the disease; (2) inhibiting the disease or arresting its development; or (3) ameliorating or causing regression of the disease or the symptoms of the disease. As understood in the art, “treatment” is an approach for obtaining beneficial or desired results, including clinical results. For the purposes of this invention, beneficial or desired results can include one or more, but are not limited to, alleviation or amelioration of one or more symptoms, diminishment of extent of a condition (including a disease), stabilized (i.e., not worsening) state of a condition (including disease), delay or slowing of condition (including disease), progression, amelioration or palliation of the condition (including disease), states and remission (whether partial or total), whether detectable or undetectable. Preferred are compounds that are potent and can be administered locally at very low doses, thus minimizing systemic adverse effects.

As used herein, the term “pharmaceutically acceptable carrier” encompasses any of the standard pharmaceutical carriers, such as a phosphate buffered saline solution, water, and emulsions, such as an oil/water or water/oil emulsion, and various types of wetting agents. The compositions also can include stabilizers and preservatives. For examples of carriers, stabilizers and adjuvants, see Martin (1975) Remington's Pharm. Sci., 15th Ed. (Mack Publ. Co., Easton).

A “subject,” “individual” or “patient” is used interchangeably herein, and refers to a vertebrate, preferably a mammal, more preferably a human. Mammals include, but are not limited to, murines, rats, rabbit, simians, bovines, ovine, porcine, canines, feline, farm animals, sport animals, pets, equine, and primate, particularly human. Besides being useful for human treatment, the present invention is also useful for veterinary treatment of companion mammals, exotic animals and domesticated animals, including mammals, rodents, and the like.

The term “administration” shall include without limitation, administration by ocular, oral, intra-arterial, parenteral (e.g., intramuscular, intraperitoneal, inhalation, transdermal intravenous, ICV, intracisternal injection or infusion, subcutaneous injection, or implant), by inhalation spray nasal, vaginal, rectal, sublingual, urethral (e.g., urethral suppository) or topical routes of administration (e.g., gel, ointment, cream, aerosol, ocular etc.) and can be formulated, alone or together, in suitable dosage unit formulations containing conventional non-toxic pharmaceutically acceptable carriers, adjuvants, excipients, and vehicles appropriate for each route of administration. The invention is not limited by the route of administration, the formulation or dosing schedule.

The term “protein”, “peptide” and “polypeptide” are used interchangeably and in their broadest sense refer to a compound of two or more subunit amino acids, amino acid analogs or peptidomimetics. The subunits may be linked by peptide bonds. In another embodiment, the subunit may be linked by other bonds, e.g., ester, ether, etc. A protein or peptide must contain at least two amino acids and no limitation is placed on the maximum number of amino acids which may comprise a protein's or peptide's sequence. As used herein the term “amino acid” refers to either natural and/or unnatural or synthetic amino acids, including glycine and both the D and L optical isomers, amino acid analogs and peptidomimetics. The peptides of the invention can include dimers and trimers of the peptides, for example a peptide can include one or several copies of RGNEF. A multimer according to the invention can either be a homomer, consisting of a multitude of the same peptide, or a heteromer consisting of different peptides.

Alternatively or in addition, the peptides can contain additional stabilizing flanking sequences. Stabilizing flanking sequences can increase the biological availability of the peptides.

Furthermore, the peptides can also encompass functionally equivalent variants or analogues of the peptides of the present invention. This includes peptides having peptides having one or more conservative or non-conservative amino acid substitutions as compared to the sequences of the peptides described herein. The substitution is preferably a conservative substitution, and does not negatively impact the biological or structural properties of the peptide (e.g., the ability to bind to TDP-43).

Functional analogues may be generated by conservative or non-conservative amino acid substitutions. Amino acid substitutions may be generally based on the relative similarity of the amino acid side-chain substituents, for example, their hydrophobicity, hydrophilicity, charge, size and the like. Thus, within the scope of the invention, conservative amino acid changes means an amino acid change at a particular position which may be of the same type as originally present; i.e. a hydrophobic amino acid exchanged for a hydrophobic amino acid, a basic amino acid for a basic amino acid, etc. Examples of conservative substitutions may include, without limitation, the substitution of non-polar (hydrophobic) residues such as isoleucine, valine, leucine or methionine for another, the substitution of one polar (hydrophilic) residue for another such as between arginine and lysine, between glutamine and asparagine, between glycine and serine, the substitution of one basic residue such as lysine, arginine or histidine for another, or the substitution of one acidic residue, such as aspartic acid or glutamic acid for another, the substitution of a branched chain amino acid, such as isoleucine, leucine, or valine for another, the substitution of one aromatic amino acid, such as phenylalanine, tyrosine or tryptophan for another. Such amino acid changes may result in functional analogues in that they may not significantly alter the overall charge and/or configuration of the peptide. Examples of such conservative changes are well-known to the skilled artisan and are within the scope of the present invention. Conservative substitution may also include the use of a chemically derivatized residue in place of a non-derivatized residue provided that the resulting peptide is a biologically functional equivalent to the peptides of the invention.

The term “isolated” or “recombinant” as used herein with respect to nucleic acids, such as DNA or RNA, refers to molecules separated from other DNAs or RNAs, respectively that are present in the natural source of the macromolecule as well as polypeptides. The term “isolated or recombinant nucleic acid” is meant to include nucleic acid fragments which are not naturally occurring as fragments and would not be found in the natural state. The term “isolated” is also used herein to refer to polynucleotides, polypeptides and proteins that are isolated from other cellular proteins and is meant to encompass both purified and recombinant polypeptides. In other embodiments, the term “isolated or recombinant” means separated from constituents, cellular and otherwise, in which the cell, tissue, polynucleotide, peptide, polypeptide, protein, antibody or fragment(s) thereof, which are normally associated in nature. For example, an isolated cell is a cell that is separated from tissue or cells of dissimilar phenotype or genotype. An isolated polynucleotide is separated from the 3′ and 5′ contiguous nucleotides with which it is normally associated in its native or natural environment, e.g., on the chromosome. As is apparent to those of skill in the art, a non-naturally occurring polynucleotide, peptide, polypeptide, protein, antibody or fragment(s) thereof, does not require “isolation” to distinguish it from its naturally occurring counterpart.

It is to be inferred without explicit recitation and unless otherwise intended, that when the present invention relates to a polypeptide, protein, polynucleotide or antibody, an equivalent or a biologically equivalent of such is intended within the scope of this invention. As used herein, the term “biological equivalent thereof” is intended to be synonymous with “equivalent thereof” when referring to a reference protein, antibody, polypeptide or nucleic acid, intends those having minimal homology while still maintaining desired structure or functionality. Unless specifically recited herein, it is contemplated that any polynucleotide, polypeptide or protein mentioned herein also includes equivalents thereof. For example, an equivalent intends at least about 70% homology or identity, or alternatively about 80% homology or identity and alternatively, at least about 85%, or alternatively at least about 90%, or alternatively at least about 95% or alternatively 98% percent homology or identity and exhibits substantially equivalent biological activity to the reference protein, polypeptide or nucleic acid. In another aspect, the term intends a polynucleotide that hybridizes under conditions of high stringency to the reference polynucleotide or its complement.

A polynucleotide or polynucleotide region (or a polypeptide or polypeptide region) having a certain percentage (for example, 80%, 85%, 90% or 95%) of “sequence identity” to another sequence means that, when aligned, that percentage of bases (or amino acids) are the same in comparing the two sequences. The alignment and the percent homology or sequence identity can be determined using software programs known in the art, for example those described in Current Protocols in Molecular Biology (Ausubel et al., eds. 1987) Supplement 30, section 7.7.18, Table 7.7.1. Preferably, default parameters are used for alignment. A preferred alignment program is BLAST, using default parameters. In particular, preferred programs are BLASTN and BLASTP, using the following default parameters: Genetic code=standard; filter=none; strand=both; cutoff=60; expect=10; Matrix=BLOSUM62; Descriptions=50 sequences; sort by =HIGH SCORE; Databases=non-redundant, GenBank+EMBL+DDBJ+PDB+GenBank CDS translations+SwissProtein+SPupdate+PIR. Details of these programs can be found at the following Internet address: ncbi.nlm.nih.gov/cgi-bin/BLAST.

“Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 30% identity or alternatively less than 25% identity, less than 20% identity, or alternatively less than 10% identity with one of the sequences of the present invention.

“Homology” or “identity” or “similarity” can also refer to two nucleic acid molecules that hybridize under stringent conditions to the reference polynucleotide or its complement.

“Hybridization” refers to a reaction in which one or more polynucleotides react to form a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these. A hybridization reaction may constitute a step in a more extensive process, such as the initiation of a PCR reaction, or the enzymatic cleavage of a polynucleotide by a ribozyme.

Examples of stringent hybridization conditions include: incubation temperatures of about 25° C. to about 37° C.; hybridization buffer concentrations of about 6×SSC to about 10× SSC; formamide concentrations of about 0% to about 25%; and wash solutions from about 4×SSC to about 8×SSC. Examples of moderate hybridization conditions include: incubation temperatures of about 40° C. to about 50° C.; buffer concentrations of about 9×SSC to about 2×SSC; formamide concentrations of about 30% to about 50%; and wash solutions of about 5×SSC to about 2×SSC. Examples of high stringency conditions include: incubation temperatures of about 55° C. to about 68° C.; buffer concentrations of about 1×SSC to about 0.1×SSC; formamide concentrations of about 55% to about 75%; and wash solutions of about 1×SSC, 0.1×SSC, or deionized water. In general, hybridization incubation times are from 5 minutes to 24 hours, with 1, 2, or more washing steps, and wash incubation times are about 1, 2, or 15 minutes. SSC is 0.15 M NaCl and 15 mM citrate buffer. It is understood that equivalents of SSC using other buffer systems can be employed.

The term “effective amount” refers to a quantity sufficient to achieve a beneficial or desired result or effect. In the context of therapeutic or prophylactic applications, the effective amount will depend on the type and severity of the condition at issue and the characteristics of the individual subject, such as general health, age, sex, body weight, and tolerance to pharmaceutical compositions. In the context of an immunogenic composition, in some embodiments the effective amount is the amount sufficient to result in the inhibition of the cytotoxic effects of TDP-43. With respect to immunogenic compositions, in some embodiments the effective amount will depend on the intended use, the degree of immunogenicity of a particular antigenic compound, and the health/responsiveness of the subject's immune system, in addition to the factors described above. The skilled artisan will be able to determine appropriate amounts depending on these and other factors.

In the case of an in vitro application, in some embodiments the effective amount will depend on the size and nature of the application in question. It will also depend on the nature and sensitivity of the in vitro target and the methods in use. The skilled artisan will be able to determine the effective amount based on these and other considerations. The effective amount may comprise one or more administrations of a composition depending on the embodiment.

### Overview

The inventors have, surprisingly, shown that the Rho guanine nucleotide exchange factor (RGNEF) has general detoxification properties and can it be used for reducing, inhibiting, preventing or treating cytotoxicity in a cell, or used increase life expectancy in a subject. As such, in one embodiment, the present disclosure provides a method for reducing, inhibiting, preventing or treating cytotoxicity in a cell, the method comprises, or alternatively consists essentially of, or yet further consists of, expressing or overexpressing in the cell a Rho guanine nucleotide exchange factor (RGNEF), or expressing or overexpressing in the cell a RGNEF analog or agonist, or expressing or overexpressing in the cell a leucine rich domain of the RGNEF, whereby the cytotoxicity in the cell is reduced, inhibited, prevented or treated. In one aspect, the cytotoxicity is TDP-43 induced.

In another embodiment, the present disclosure is a use of a Rho guanine nucleotide exchange factor (RGNEF) or a RGNEF analog or agonist or a leucine rich domain of the RGNEF for reducing, inhibiting, preventing or treating cytotoxicity in a cell. In one aspect, the cytotoxicity is TDP-43 induced.

In another embodiment, the present invention is a method of shifting the survival curve of a subject and thereby increasing life expectancy of the subject, the method comprises, or alternatively consists essentially of, or yet further consists of, administering to the subject a physiologically effective amount of a peptide comprising a Rho guanine nucleotide exchange factor (RGNEF), or a RGNEF analog or agonist, or a leucine rich domain of the RGNEF, or expressing or overexpressing RGNEF or a RGNEF analog or agonist, or a leucine rich domain of RGNEF in the subject.

In another embodiment, the present disclosure relates to a use of a peptide comprising a Rho guanine nucleotide exchange factor (RGNEF), a RGNEF analog or agonist, or a leucine rich domain of RGNEF, or expressing or overexpressing RGNEF or a RGNEF analog or agonist, or a leucine rich domain of the RGNEF for shifting the survival curve of a subject and thereby increasing life expectancy of the subject.

The inventors have, surprisingly, shown that inhibition of TDP-43 by an antagonist decreases TDP-43 toxicity. The inventors have, surprisingly, shown that both the full-length Rho guanine nucleotide exchange factor (RGNEF) and a smaller N-terminus fragment (the leucine-rich domain) can inhibit the toxicity of TDP-43. Importantly, over 98% of all ALS cases regardless of inherited or sporadic origin demonstrate intracellular aggregates of TDP-43 within the affected neuronal populations and experimental models of altered TDP-43 expression or metabolism give rise to cell death; it was discovered that RGNEF plays as critical regulator of motor neuron death that is the hallmark of ALS. By observing that RGNEF, or even a critical fragment of the protein, can modify the toxic phenotype induced by TDP-43 in vivo a novel therapeutic is disclosed herein for this uniformly fatal disorder.

### Method

The methods of the present invention are useful to inhibit cytotoxicity, including the degeneration of neurons. The method can be used as a first line, a second line or a third line therapy for a patient.

In one embodiment, a method of inhibiting cytotoxicity, including the degeneration of neurons comprises, or alternatively consists essentially of, or yet further consists of, administering an effective amount of an antagonist of TDP-43 to a subject in need.

In one embodiment, the present disclosure provides a method for inhibiting neuron degeneration, or inhibiting motor neuron degeneration, or treating a condition associated with TDP-43 toxicity, or treating ALS in a patient comprising, or alternatively consisting essentially of, or yet further consisting of administering to the patient an effective amount of an agent that binds and inhibits TDP-43 or an antagonist of TDP-43, thereby inhibiting the motor neuron degeneration, or treating the condition associated with TDP-43 toxicity, or treating ALS in the patient.

Any suitable route of administration is acceptable, and can be determined by the treating physician. Non-limiting examples include intravenously or by inhalation therapy.

The method can be repeated with varying cycles, e.g., two, three, four, five, six, seven, eight or more, and can be used as a maintenance therapy for a patient. For maintenance therapy, the time between the first and second therapy is about 21 days to 35 days there between, or alternatively every 26 days to 30 days there between or alternatively about every 5 to 6 weeks.

In a further aspect, the method further comprises, or alternatively consists essentially of, or yet further consists of, administering an effective amount of a second agent that treats neuron degeneration or enhances the effect of the antagonist agents that inhibit TDP-43 toxicity. Non-limiting examples of are described herein, e.g., one or more of riluzole, baclofen, diazepam, gabapentin arimoclomol, trihexyphenidyl.

In embodiments, the present disclosure relates to a use of a TDP-43 antagonist to inhibit cytotoxicity, including the degeneration of neurons. In other embodiments, the present disclosure relates to a use of a TDP-43 antagonist in the treatment of a condition associated with TDP-43 toxicity, or for treating ALS.

In embodiments, the present disclosure relates to a use of a TDP-43 antagonist in the manufacture of a medicament to inhibit cytotoxicity, including the degeneration of neurons. In other embodiments, the present disclosure relates to a use of a TDP-43 antagonist in the manufacture of a medicament for the treatment of a condition associated with TDP-43 toxicity, or for treating ALS.

### TDP-43 Antagonists

In one embodiment the TDP-43 antagonists that may be used in the present invention include TDP-43 ligands that bind TDP-43 thereby inhibiting or cancelling the cytotoxicity of TDP-43. In another embodiment the TDP-43 antagonist may be a substance or molecule that does not bind to TDP-43, but that nevertheless reduces the cytotoxic effects of TDP-43. For example, in one embodiment, a TDP-43 antagonist includes a substance or molecule that increases the expression of RGNEF protein.

TDP-43 antagonist may, for example, be a peptide comprising or alternatively consisting essentially of, or yet further consisting of RGNEF or comprising or alternatively consisting essentially of, or yet further consisting of functionally equivalent variants, agonists or analogues of RGNEF. Such peptide, variants, agonists and analogues, and nucleic acids encoding these peptides, variants, agonists and analogues, may be used to inhibit TDP-43 activity.

In one embodiment, the TDP-43 antagonist comprises a full length RGNEF or alternatively consists essentially of, or yet further consists of a full length RGNEF. In another embodiment the TDP-43 antagonist comprises or alternatively consists essentially of, or yet further consists of a leucine-rich RGNEF domain. In another embodiment the TDP-43 antagonist comprises or alternatively consists essentially of, or yet further consists of a fragment of RGNEF that effectively inhibits the cytotoxicity of TDP-43.

In this document, a “peptide comprising RGNEF” refers to a polypeptide or peptide or protein that contains one or more copies of the full RGNEF sequence. A “peptide comprising a leucine-rich RGNEF domain” refers to a polypeptide, peptide or protein that contains one or more copies of the leucine rich RGNEF domain.

In one embodiment, the TDP-43 antagonist may be a low molecular weight antagonist. Specific examples of low molecular weight TDP-43 antagonists that may be used in the methods and compositions of the present invention may include glutathione monoethyl ester (ref. PMID: 28818672); N-Benzothiazolyl-2-Phenyl Acetamide derivatives (theoretical; ref. PMID: 28155653); nilotinib and bosutinib (ref. PMID: 27507246); IGS-2.7 and IGS-3.27 (ref. PNID: 27138926); PHA767491 (ref. PMID: 23424178, to name a few.

In another embodiment, the antagonist of TDP-43 may include or consist of an antibody directed against an epitope or epitopes of TDP-43, in such a way that said antibody impairs the neural toxicity of TDP-43, or an antibody against a target that affects the cytotoxic effects of TDP-43, or an antibody that increases the expression of RGNEF.

Antibodies may be raised according to any known methods by administering an appropriate antigen or epitope to a host animal (e.g., from pigs, cows, horses, rabbits, goats, sheep, and mice, among others). Various adjuvants known in the art may be used to enhance antibody production. In addition, plants and bioreactors may also be used to enhance antibody production. Although antibodies useful in practicing the invention may be polyclonal, monoclonal antibodies may be preferred. Monoclonal antibodies against may be prepared and isolated using any technique that provides for the production of antibody molecules by continuous cell lines in culture. Techniques for production and isolation include the hybridoma technique originally described by [40]; the human B-cell hybridoma technique (Cote et al., 1983); and the EBV-hybridoma technique [41]. Alternatively, techniques described for the production of single chain antibodies (see, e.g., U.S. Pat. No. 4,946,778) may be adapted to produce antibodies of the present invention. TDP-43 antagonists useful in practicing the present invention also include antibody fragments including but not limited to F(ab′) 2 fragments, which can be generated by pepsin digestion of an intact antibody molecule, and Fab fragments, which can be generated by reducing the disulfide bridges of the F(ab′) 2 fragments. Alternatively, Fab and/or scFv expression libraries can be constructed to allow rapid identification of fragments having the desired specificity to a target.

In general, cells actively expressing the protein are cultured or isolated from tissues and the cell extracts isolated. The extracts or recombinant protein extracts, containing the TDP-43, are injected in Freund's adjuvant into mice. After being injected 9 times over a three week period, the mice spleens are removed and resuspended in phosphate buffered saline (PBS). The spleen cells serve as a source of lymphocytes, some of which are producing antibody of the appropriate specificity. These are then fused with a permanently growing myeloma partner cell, and the products of the fusion are plated into a number of tissue culture wells in the presence of a selective agent such as HAT. The wells are then screened to identify those containing cells making useful antibody by ELISA. These are then freshly plated. After a period of growth, these wells are again screened to identify antibody-producing cells. Several cloning procedures are carried out until over 90% of the wells contain single clones which are positive for antibody production. From this procedure a stable lines of clones is established which produce the antibody. The monoclonal antibody can then be purified by affinity chromatography using Protein A or Protein G Sepharose.

In one embodiment of the invention, the inhibitor of TDP-43 may be a siRNA, a ribozyme, or an antisense oligonucleotide

Antisense oligonucleotides may be used in the methods of the present invention to, for example, increase the levels of RGNEF in the cell.

Consequently, the present invention provides a method of inhibiting the effects of TDP-43 comprising or consisting or consisting essentially of administering an effective amount of an antisense oligonucleotide that is complimentary to a nucleic acid sequence that inhibits the expression of RGNEF to an animal in need thereof.

The antisense nucleic acid molecules may be constructed using chemical synthesis and enzymatic ligation reactions using procedures known in the art. The antisense nucleic acid molecules of the invention or a fragment thereof, may be chemically synthesized using naturally occurring nucleotides or variously modified nucleotides designed to increase the biological stability of the molecules or to increase the physical stability of the duplex formed with mRNA or the native gene e.g. phosphorothioate derivatives and acridine substituted nucleotides. The antisense sequences may be produced biologically using an expression vector introduced into cells in the form of a recombinant plasmid, phagemid or attenuated virus in which antisense sequences are produced under the control of a high efficiency regulatory region, the activity of which may be determined by the cell type into which the vector is introduced.

Small inhibitory RNA (siRNA) is a form of gene silencing triggered by double-stranded RNA (dsRNA). In siRNA sequence-specific, post-transcriptional gene silencing in animals and plants may be initiated by double-stranded RNA (dsRNA) that is homologous in sequence to the silenced gene. A siRNA (small interfering RNA) is designed to target and thus to degrade a desired mRNA (in this case encoding mRNA that inhibits the expression of RGNEF) in order not to express the encoded protein (in this case TDP-43). Methods relating to the use of siRNA (or RNA interference) to silence genes in C. elegans, Drosophila, plants, and mammals are known in the art [42-52, WO0129058; WO9932619, the disclosures of which are incorporated herein in their entirety].

Ribozymes may also function as inhibitors of TDP-43 expression for use in the present invention. Ribozymes are enzymatic RNA molecules capable of catalyzing the specific cleavage of RNA. The mechanism of ribozyme action involves sequence specific hybridization of the ribozyme molecule to complementary target RNA, followed by endonucleolytic cleavage. Engineered hairpin or hammerhead motif ribozyme molecules that specifically and efficiently catalyze endonucleolytic cleavage of TDP-43 mRNA sequences are thereby useful within the scope of the present invention. Specific ribozyme cleavage sites within any potential RNA target are initially identified by scanning the target molecule for ribozyme cleavage sites, which typically include the following sequences, GUA, GuU, and GUC. Once identified, short RNA sequences of between about 15 and 20 ribonucleotides corresponding to the region of the target gene containing the cleavage site can be evaluated for predicted structural features, such as secondary structure, that can render the oligonucleotide sequence unsuitable. The suitability of candidate targets may also be evaluated by testing their accessibility to hybridization with complementary oligonucleotides, using, e.g., ribonuclease protection assays. Ribozimes may also be engineered to target RNA that inhibits the expression of RGNEF in a cell.

### Compositions

In one embodiment, the present invention provides for a composition for treating a condition associated with TDP-43 cytotoxicity, the composition includes any one of the TDP-43 antagonist previously described. In aspects, the composition further includesa pharmaceutically acceptable carrier. In aspects of the present invention, the composition is used for treating, preventing or minimizing complications associated with TDP-43 cytotoxicity.

The present inventors have identified novel compositions and methods for inhibiting TDP-43 cytotoxicity.

One embodiment of the present invention further encompasses pharmaceutical compositions comprising a TDP-43 antagonist for administration to subjects in a biologically compatible form suitable for administration in vivo. By “biologically compatible form suitable for administration in vivo” is meant a form of the substance to be administered in which any toxic effects are outweighed by the therapeutic effects. Administration of a therapeutically active amount of the pharmaceutical compositions of the present invention, or an “effective amount”, is defined as an amount effective at dosages and for periods of time, necessary to achieve the desired result of eliciting an immune response in a human. A therapeutically effective amount of a substance may vary according to factors such as the disease state/health, age, sex, and weight of the recipient, and the inherent ability of the particular polypeptide, nucleic acid coding therefor, or recombinant virus to elicit a desired immune response. Dosage regimen may be adjusted to provide the optimum therapeutic response. For example, several divided doses may be administered daily or on at periodic intervals, and/or the dose may be proportionally reduced as indicated by the exigencies of the therapeutic situation. The amount of TDP-43 antagonist for administration will depend on the nature of the TDP-43 antagonist, the route of administration, time of administration and varied in accordance with individual subject responses. Suitable administration routes may be intramuscular injections, subcutaneous injections, intravenous injections or intraperitoneal injections, oral and intranasal administration. In a preferred embodiment, the administration route may be intravenous injection.

As such, one embodiment of the present invention may be administering the TDP-43 antagonist by injection. Another embodiment of the present invention may be administering the TDP-43 antagonist intravenously with a carrier in the form of normal saline solution. Another embodiment of the present invention may be administering the TDP-43 antagonist by liposome or nanoparticle delivery. Another embodiment of the present invention may be administering the TDP-43 antagonist via an implantable device capable of controlled release of the TDP-43 antagonist. For example US Pat. Appl. No. 20050208122 (which is incorporated herein by reference) discloses a biodegradable biocompatible implant for controlled release of therapeutically active agents, which may be used to administer the TDP-43 antagonist according to the embodiments of the present invention.

The compositions described herein may be prepared by per se known methods for the preparation of pharmaceutically acceptable compositions which may be administered to subjects, such that an effective quantity of the active substance (i.e. TDP-43 antagonist) is combined in a mixture with a pharmaceutically acceptable vehicle. Suitable vehicles are described, for example, in “Handbook of Pharmaceutical Additives” (compiled by Michael and Irene Ash, Gower Publishing Limited, Aldershot, England (1995)). On this basis, the compositions include, albeit not exclusively, solutions of the substances in association with one or more pharmaceutically acceptable vehicles or diluents, and may be contained in buffered solutions with a suitable pH and/or be iso-osmotic with physiological fluids. In this regard, reference can be made to U.S. Pat. No. 5,843,456.

Pharmaceutical acceptable carriers are well known to those skilled in the art and include, for example, sterile saline, lactose, sucrose, calcium phosphate, gelatin, dextrin, agar, pectin, peanut oil, olive oil, sesame oil and water.

Furthermore the pharmaceutical composition according to the invention may comprise one or more stabilizers such as, for example, carbohydrates including sorbitol, mannitol, starch, sucrose, dextrin and glucose, proteins such as albumin or casein, and buffers like alkaline phosphates.

A major advantage of this invention includes protecting cells such as neurons or motor neurons. As such, in one embodiment, the present invention is directed to pharmaceutical compositions comprising a TDP-43 antagonist for promoting neuron protection.

The above disclosure generally describes the present invention. A more complete understanding can be obtained by reference to the following specific examples. These Examples are described solely for purposes of illustration and are not intended to limit the scope of the invention. Changes in form and substitution of equivalents are contemplated as circumstances may suggest or render expedient. Although specific terms have been employed herein, such terms are intended in a descriptive sense and not for purposes of limitation.

### Life Expectancy

The life expectancy of many subjects, including mammals, is now known. For example, while the majority of humans die before age 85, the maximum life span of humans is thought to be between 110 and 120 years. Likewise for dogs, while larger breeds of dogs grow older faster, in 7-10 years, smaller dogs become old between 10 and 13 years (Deeb, B. J., and N. S. Wolf. Studying longevity and morbidity in giant and small breeds of dogs. Veterinary Medicine (Supplement). July, 1994. pp. 702-713).

In accordance with the present disclosure, it has been demonstrated that the fruit fly survival curve can be shifted in a favorable fashion, increasing the fruit fly's life expectancy, if the animal carries at least one copy of RGNEF (see FIG. 11).

In the case of mammals, the administration of RGNEF may begin with about 5% of the normal life span of the subject completed. In another embodiment, the administration may begin with about 10%, 15%, 20%, 25%, 30%, 35%, 40%, 45%, 50% 55% 60%, 65%, 70%, 75%, 80%, 85%, 90% or 95% of the normal life span of the subject completed. As such, in embodiment, the present disclosure is a method of shifting the survival curve of a subject and thereby increasing life expectancy of the subject, comprising administering to the subject a physiologically effective amount of a peptide comprising or consisting or RGNEF, or a RGNEF analog or agonist, or a peptide comprising or consisting of the leucine rich domain of RGNEF, or expressing or overexpressing RGNEF or a RGNEF analog or agonist or a leucine rich domain of RGNEF in the subject. In another aspect a method of shifting the survival curve of a subject comprises administering to the subject a physiologically effective amount of a molecule or substance that increases the expression of RGNEF in the subject.

In one embodiment, the present invention relates to a method of treating, inhibiting or reducing cytotoxicity in a cell. The method, in one embodiment, includes contacting, transferring to, expressing or overexpressing in said cell a peptide comprising or consisting or a RGNEF, or a a RGNEF analog or RGNEF agonist or a peptide comprising or consisting of a leucine rich domain of RGNEF. The cell suffering from cytotoxicity can be genetically modified to overexpress the RGNEF and/or the RGNEF analog or RGNEF agonist or the leucine rich domain of RGNEF according to engineering, including genetic engineering, techniques known in the art.

Engineered cells, including genetically engineered cells, expressing or overexpressing RGNEF can also be used in cell therapy for treating a subject of a disorder, such as a TDP-43 associated disorder, or to shifting the survival curve of a subject.

In one embodiment, the present disclosure relates to a use of of a peptide comprising or consisting of RGNEF, or a RGNEF analog or agonist, or a peptide comprising or consisting of a leucine rich domain of RGNEF, or expressing or overexpressing RGNEF or a RGNEF analog or agonist or a leucine rich domain of RGNEF, for shifting the survival curve of a subject, thereby increasing life expectancy of the subject.

### Experimental

### Example 1

### Materials and Methods

### 1. Constructs

For the generation of constructs used in the in vitro cytoprotection studies, in addition to the construct pcDNA-RGNEF-myc expressing a myc-tagged full length RGNEF protein, we generated six RGNEF constructs with deletions in several domains by standard molecular biology procedures. PCR reactions were performed using Pfu DNA polymerase and the products were cloned into the vector pcDNA 3.1/myc-His A (Invitrogen) for expression. All constructs were confirmed by sequencing. The primers used in PCR to generate deletion constructs of RGNEF are as shown below in Table 1:

TABLE 1

| Name | Primer | Sequence (5′ → 3′) |
|---|---|---|
| RGNEF-ΔGEF | 1) Forward (I) | attggtaccatggagttgagctgcagcgaa |
| | | (SEQ ID NO: 1) |
| | 2) Reverse (I) | taaggaggtgtttgatgacatccttctcctgc |
| | | (SEQ ID NO: 2) |
| | 3) Forward (II) | tgtcatcaaacacctccttattaaacctgaccca |
| | | (SEQ ID NO: 3) |
| | 4) Reverse (II) | agactcgagcaccttgaggtctacttgatgtt |
| | | (SEQ ID NO: 4) |
| RGNEF-ΔRBD | 5) Forward | attggtaccatggagttgagctgcagcgaa |
| | | (SEQ ID NO: 5) |
| | 6) Reverse | caactggaggggctctagatggacgtcctc |
| | | (SEQ ID NO: 6) |
| RGNEF-NH₂ | 7) Forward | attggtaccatggagttgagctgcagcgaa |
| | | (SEQ ID NO: 7) |
| | 8) Reverse | tttctcgagtttgatgacatccttctcctgcctattac |
| | | (SEQ ID NO: 8) |
| RGNEF-COOH | 9) Forward | ataggtaccatgagacaggatgtcatttttgagcta |
| | | (SEQ ID NO: 9) |
| | 10) Reverse | agactcgagcaccttgaggtctacttgatgtt |
| | | (SEQ ID NO: 10) |
| RGNEF-NH₂-ΔL- | 11) Forward | ataggtaccatgattcactcatcggaaacgct |
| rich | | (SEQ ID NO: 11) |
| | 12) Reverse | tttctcgagtttgatgacatccttctcctgcctattac |
| | | (SEQ ID NO: 12) |

*Restriction enzyme sequences are underlined. XhoI = ctcgag; KpnI = ggtacc.
**RGNEF-ΔGEF is a fusion protein made by using two sets of primers, where primer 2 and 3 exhibit complementary sequences overlap.

For the generation of TDP-43 and LeuR constructs used in the metabolic stressor studies, PCR reactions were performed using Phusion High-Fidelity DNA Polymerase (Thermo Scientific) and the products were cloned into the vectors pcDNA 3.1/myc-His A (Invitrogen) or pcDNA 3.1 (Invitrogen) respectively. A flag tag was added in the 5′ extreme of the coding sequence of LeuR region and full length RGNEF for later detection using anti-flag antibodies. All constructs were confirmed by sequencing.

### 2. Cell Lines and Transfections

For in vitro studies evaluating the ability of RGNEF to protect against a stressor, Human embryonic kidney cells (HEK293T cells, Dharmacon, Cat. #HCL4517) were maintained in Dulbecco's modified Eagle's medium (DMEM; Life Technologies Inc., #11995-065) supplemented with 10% of fetal bovine serum (FBS; Gibco, Life Technologies Inc.), 100 U/ml penicillin/streptomycin (Life Technologies Inc., Burlington ON, #15140-122) and 5 μg/ml of plasmocin (Life Technologies Inc., #ant-mpt)). Cells were grown in a water-jacketed, 37° C., 5% CO₂ incubator (VWR, #10810-878).

Transient transfections were performed using Lipofectamine 2000 (Life Technologies) in 6-well plates according to the manufacturer's protocol. All experiments were performed 48 hours post-transfection.

For in vitro studies of the induction of TDP-43 cytosolic aggregates following a metabolic stress, HEK293T and SH-5YSY cells (ATCC) were maintained in 25 mM glucose, 1 mM pyruvate Dulbecco's modified Eagle's medium (Gibco—Life technologies) containing 100 U/ml penicillin, 100 U/ml streptomycin (Gibco—Life technologies), 5 μg/ml plasmocin (InvivoGen), and 10% fetal bovine serum (Gibco—Life technologies). HEK293T cells maintained 7 days under metabolic stress (HG+lactate) or under control conditions (HG) were seeded onto coverslips previously treated with attachment factor (Gibco) in 6-well plates at 250,000 cells/ml 24 hours before transfection. Transfections were performed using Lipofectamine 2000 (ThermoFisher Scientific) at 70% of confluency using 2.5 μg of DNA in a ratio (μg) DNA:(μl) lipofectamine 1:2.5. Cells were maintained under metabolic stress for 48 hours after the transfection. Then, cells in the coverslips were fixed in 4% paraformaldehyde in PBS for 15 min and processed for immunofluorescence. For the cross-linking-immunoprecipitation experiment transfections were performed using the same protocol, but using 6-well plates without coverslips and without metabolic stress. Cell viability was quantified using either MTT assay (Thiazol Blue Tetrazolium Bromide; Sigma-Aldrich) or trypan blue exclusion assay (Life Technologies). To perform these assays, HEK293T cells were seeded in 96-well plates at 10,000 cells/ml 24 hours post-transfection. The MTT assay was performed after an additional 24 hours by incubating cells with MTT (1 mg/ml) for 1 hour during the stress treatment, after which the formazan formed by mitochondrial reduction was quantified using a microplate reader. The percent survival was expressed as [Percent Survival=(Stressed Cell Absorbance/Untreated Cell Absorbance)×100] where “Stressed Cell Absorbance” is the mean absorbance value calculated for cells that had been exposed to a stress condition and “Untreated Cell Absorbance” is the mean absorbance value for corresponding cells that had only had their media replaced. For trypan blue exclusion, cells were incubated for 1 minute with trypan blue solution (1:100), and then the ratio of live to total (live and dead) cells, or the “ratio of surviving cells” was determined by counting with a hemocytometer under a light microscope. Cell cytotoxicity was assayed 48 hours after transfection with the RGNEF-myc expressing construct or the empty plasmid as control. The level of LDH (lactate dehydrogenase) released into the media from damaged cells and accounting for cellular cytotoxicity and cytolysis, was measured using the CytoTox 96 Non-Radioactive Cytotoxicity Assay kit (Promega, Madison, WI) according to the manufacturer's protocol.

### 3. Stress Conditions

Oxidative or osmotic stress conditions. RGNEF overexpressing cells were exposed to an oxidative or osmotic stress using 500 μM sodium arsenite (Sigma-Aldrich, Oakville, ON) or 400 mM sorbitol (Sigma-Aldrich), respectively.

Metabolic stress conditions. To induce a metabolic stress, cells were incubated with 30 mM lactate (DL-Lactic acid sodium salt, Sigma-Aldrich) in 10% FBS DMEM containing 25 mM glucose (high glucose-HG) and 1 mM pyruvate or 0.6 mM glucose (low glucose-LG) media (glucose given by the FBS present in the media).

### 4. Immunofluorescence and Confocal Microscopy

Oxidative or osmotic stress conditions. Cells were seeded onto coverslips contained in a 6-well plate at 250,000 cells/ml 24 hours after transfection. Then, after another 24 hours, cells were exposed to either oxidative or osmotic stress. Coverslips were then fixed in 4% paraformaldehyde for 15 minutes followed by permeabilization with 0.2% Triton X-100 for 10 minutes. Aldehyde groups were quenched using 50 mM ammonium chloride for 30 minutes. Following blocking with 8% bovine serum albumin for 1 hour at room temperature, cells were incubated with primary and secondary antibodies for 1 hour each. Nuclei were stained with Hoechst (1 μg/mL) and cells then visualized by confocal microscopy (LSM 510 META; Carl Zeiss Canada Ltd., Toronto, ON) with AIM software (version 4.2; Carl Zeiss Canada Ltd.). Antibodies used in immunofluorescence microscopy are listed in Table 2:

TABLE 2
Immunofluorescence antibodies

| | | | | | Secondary |
|---|---|---|---|---|---|
| Name | Species | Cat# | Dilution | Manufacturer | Antibody |
| c-Myc | Mouse | CLH102AP | 1:250 | Cedarlane | Alexa donkey or |
| (monoclonal) | | | | (Burlington, ON, | goat α-Mouse 488, |
| | | | | Canada) | Life Technologies |
| | | | | | (dilution: 1:800) |
| Staufen-1 | Rabbit | AB5781 | 1:100 | Millipore | Alexa goat α- |
| (polyclonal) | | | | Canada Ltd. | rabbit, 555, Life |
| | | | | (Etobicoke, ON, | Technologies |
| | | | | Canada) | (dilution: 1:800) |
| TIA-1 | Goat | 3540-100 | 1:100 | Biovision | Alexa donkey α- |
| (polyclonal) | | | | | goat 546, Life |
| | | | | (Edmonton, AB, | Technologies |
| | | | | Canada) | (dilution: 1:800) |
| RGNEF | Goat | MM-0193-P | 1:100 | MediMabs | Alexa donkey α- |
| (polyclonal) | | | | (Montreal, QC, | goat 488, Life |
| | | | | Canada) | Technologies |
| | | | | | (dilution: 1:800) |
| TDP-43 | Rabbit | 10782-2-AP | 1:500 | Proteintech | Alexa goat α- |
| (polyclonal) | | | | (Chicago, IL, | rabbit, 555, Life |
| | | | | USA) | Technologies |
| | | | | | (dilution: 1:800) |

Metabolic stress conditions. Coverslips with fixed cells were permeabilized with 0.2% Triton X-100 for 10 min. Aldehyde groups were quenched using 50 mM ammonium chloride for 30 min. Then, cells were blocked with 8% bovine serum albumin for 1 hour at room temperature. The following primary antibodies were used: mouse anti-myc (1:250; Cedarlane), mouse anti-flag (1:400; Sigma), rabbit anti-TDP-43 (1:500; Proteintech), mouse anti-TDP-43 (1:500; Proteintech), rabbit anti-RGNEF (1:100; Abcam), rabbit anti-FUS/TLS (1:100; Proteintech), rabbit anti-Sirtl (1:50; Santa Cruz Biotechnology), mouse anti-Nuclear Pore Complex Protein (NPCP) (1:500; BioLegend), rabbit anti-SOD1 (1:200; Enzo Life sciences), mouse anti-Poly A Binding Protein (PABP) (1:500; Abcam), or mouse anti-COX IV (1:125; ThermoFisher Scientific). Primary antibodies were detected using secondary antibodies conjugated to AlexaFluor 488 or Alexa Fluor 555 (1:800; Invitrogen). Nuclei were stained with Hoechst (1 μg/mL).

Cells and tissues from immunofluorescence experiments were visualized by scanning confocal microscopy (Leica TCS SP8) or super resolution confocal microscope (VT-iSIM High Speed Super Resolution Microscope-Quorum Technologies— with a spatial resolution of 125 nm laterally and 350 nm axially). The 3D reconstruction analysis of set of confocal images were performed using the Leica 3D analysis tool from LAS X software. Intensity Correlation Analysis (Li et al, 2004) using ImageJ software was performed to obtain the co-localization images. The co-localized pixels are shown as PDM (Product of the Differences from the Mean) images. PDM=(red intensity-mean red intensity)×(green intensity-mean green intensity). In the co-localization images, blue color indicates a low level of co-localization while yellow and white indicate a high degree of co-localization.

### 5. Immunoprecipitation

50 μl of recombinant Protein G-Sepharose 4B conjugated beads (Invitrogen #10-1241) were added to 1 ml of NP40 substitute lysis buffer (IGEPAL-CA630, Sigma-Aldrich, #18896-100ML) in 1×TBS solution (1% NP40 substitute, 1× protease inhibitor cocktail in 1×TBS). 1 μg of IP antibody was added to the solution and agitated at 4° C. for 30 mins. Following incubation, the NP40 buffer was removed and beads were blocked with 8% BSA dissolved in 1×TBS solution for 1 hour and 30 minutes. Beads were then incubated with 500 μg of protein lysate and agitated overnight at 4° C. The following day, beads were washed first in 0.5% TritonX-100 with 1% NP40 buffer in 1×TBS, then 1% NP40 Buffer in 1×TBS, and then 1×TBS each with a 12,000×g centrifugation at 4° C. for 2 minutes between each wash. After the final wash, the supernatant was removed, and 30 μl of 1×TBS were added, with 15 μl of loading buffer (0.08 M Tris-Base, 10% SDS, bromophenol blue, 50% glycerol, 2.5% beta-mercaptoethanol pH 7.4). Samples were then heated at 65° C. for 10 minutes then centrifuged for 2 minutes at 12,000×g at room temperature.

### 6. Transgenic Fly Development

For generation of transgenic flies, the coding region of TDP-43 wt and flag-LeuR were cloned in the pTW-UASt vector (Drosophila Genomics Resource center). The UAS-TDP-43 wt and UAS-LeuR transgenic lines were generated by germline transmission (BestGene) (Droppelmann et al., unpublished results). GMR-GAL4, D42-GAL4, and elav-GAL4 drivers fly lines were obtained from the Bloomington Stock Center (Indiana University, Bloomington, Indiana, USA.). (Table 3). Stocks and crosses were cultured according to standard procedures and on standard fly food (Water, Yeast, Soy fluor, Yellow Cornmeal, Agar, Light Corn Syrup, Propionic acid). Crosses were raised on 25° C. and 70% humidity at a 12 hour day/night cycle. The Gal4 drivers used in this study are shown in Table 3.

TABLE 3

| | Stock | | | | |
|---|---|---|---|---|---|
| Name | # | Symbol | Chr. | Expression | Genotype |
| GMR | 1104 | P{GAL4- | 2 | Eye* | w[*]; P{w[+mC] = GAL4- |
| | | ninaE.GMR} 12 | | | ninaE.GMR} 12 |
| D42 | 8816 | P{GawB}D42 | 3 | Motor | w[*]; |
| | | | | Neuron | P{w[+mW · hs] = GawB}D42 |
| ELAV | 458 | P{GawB}elav[C | 1 | Pan- | P{w[+mW · hs] = GawB} elav[C |
| | | 155] | | Neuronal** | 155] |

The genotype for single transgenic flies were: {+/+;UAS-LeuR, +CyO} and {+/+; +Sb, UAS— TDP-43 wt}. CyO is the symbol for a curly wing phenotype and Sb is the symbol for stubby hair follicle phenotype. A total of 9 lines with different insertions of each transgenic group were created using P-element mediated transformation. Having 9 lines helped control for the effects of random insertion that may be lethal or produce any phenotypic effect regardless of transgenic protein expression. Two lines were chosen of each transgenic insertion to control for this effect and to compare relative expression effects. Two lines of the leucine-rich domain transgenic flies (L2 and L4) which contain the gene locus on the second chromosome and two TDP-43 transgenic fly lines, containing the gene on the 3^{rd} chromosome were chosen (T6 and T9). Having the genes on separate chromosomes allowed for homozygosity of both genes and the removal of all balancing markers when generating the double transgenic fly line ({+/+; UAS-LeuR; UAS-TDP-43}).

Final progeny of the balanced lines for the leucine-rich domain and TDP-43 were crossed to generate the double transgenic line. The offspring that had no phenotypic markers present were chosen as that meant they were homozygous for both proteins on chromosome 2 and 3 {+/+;LeuR;TDP-43}. These flies were then run through analysis alone or crossed to drivers that drove tissue specific expression of the transgene. For genomic analysis and protein and RNA expression, whole flies were anaesthetized in a 1.5-ml microcentrifuge tube placed on ice. If not used immediately, flies were retrieved after having been stored at −80° C. and placed in a fresh tube. Functional assays for drosophila

Negative Geotaxis Assay (Climbing Assay). F1 male progeny of transgenic flies crossed with elav and D42 driver fly lines were collected. The following day, flies were transferred to a graduated cylinder divided into 4 vertical quadrants and sealed in with parafilm. Flies were tapped to the bottom of the cylinder and the number of flies present in each quadrant was recorded at 10 s and 20 s. Measurements were repeated a total of 4 times every 5 days for 30 days. Climbing index was calculated using the formula:

$$Climbing Index = ( ( Quadrant * 1 ) + ( Quadrant 2 * 2 ) + ( Quadrant 3 * 3 ) + ( Quadrant 4 * 4 ) ) / Total number of files$$

Lifespan Assay. F1 male progeny of transgenic flies crossed with Elav and D42 driver fly lines, as well as driver lines alone and non-expressing transgenic lines, were collected. Flies were raised in an incubator set to 25° C. at 70% humidity with controlled day/night cycles in vials. The number of dead and live flies were counted every 5 days until no live flies remained. The data was analyzed using Kaplan-Meier estimator.

Eye characterization. Single transgenic fly lines (LeuR-2, LeuR-4, TDP-43-6, TDP-43-9) and double transgenic fly lines (LeuR-2;TDP-43-6, LeuR-2;TDP-43-9) were crossed with the GMR-gal4 driver line for a total of 3 unique crosses. 5 male progeny from each cross were collected. Flies were on a 7 day schedule anaesthetized with CO₂ and placed in a petri dish sealed with parafilm. Flies were then photographed using a Nikon light microscope and returned to their vials once per week for up to 7 weeks or until death.

### 7. Statistical Analysis

The statistical analyses were performed with GraphPad Prism software using one-way or two-way ANOVA with Tukey post-hoc analysis to obtain exact p values. Data were expressed as mean±SEM. Data was judged to be statistically significant when p<0.05. For the drosophila studies, Kaplan-Meier estimate analysis was used to compare variance in lifespan curves. One-way ANOVA with multiple comparisons tests were run for RING climbing assay comparisons and qPCR analysis.

### Results

When HEK 293T cells were subjected to an oxidative or osmotic stress, we observed that full length RGNEF could protect the cells from injury (FIGS. 3A and 3B) (Cheung et al., 2017).

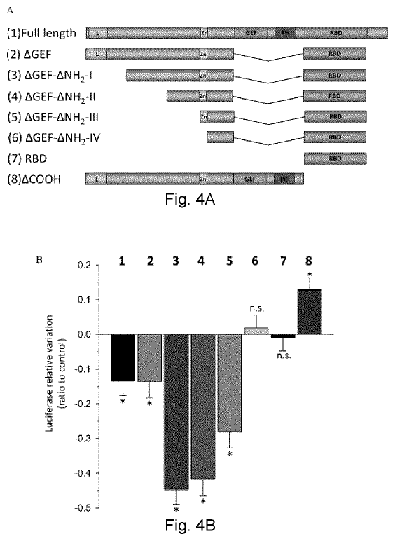

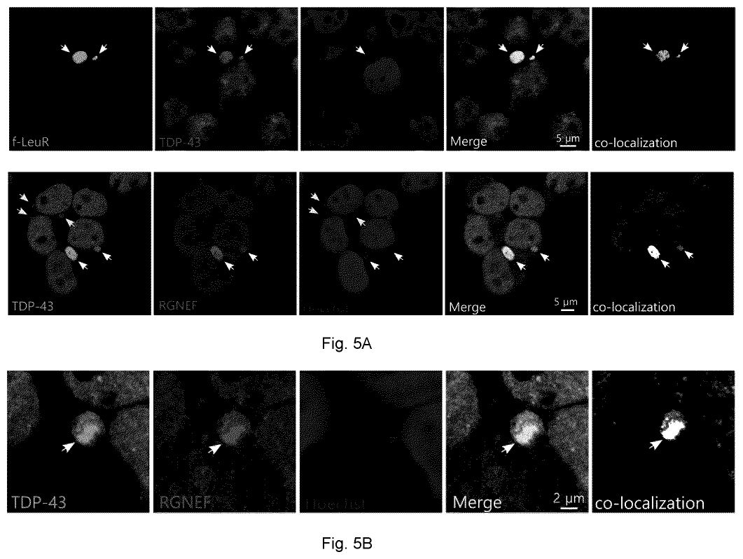

We demonstrate that the leucine rich domain of RGNEF located within the N-terminus domain is critical for protecting cells in the presence of osmotic stress—a form of cellular injury that is believed to be critical to the pathobiology of ALS. We have shown that deletion of the leucine-rich domain from full length RGNEF significantly increases the ability of RGNEF to destabilize NEFL mRNA (FIG. 4B). We have also shown that HEK293T cells subjected to metabolic stress (lactate overload) form NCIs composed of endogenous TDP-43 and that these inclusions also contain RGNEF, including the critical N-terminus domain of RGNEF (FIGS. 5A-5B). This is an important observation that the pathology of degenerating motor neurons as discussed earlier can be replicated in vitro using metabolic stress (FIGS. 5A and 5B). It also supports the concept that both TDP-43 and RGNEF are tightly integrated in the pathobiology of ALS, a concept that we have explored further by demonstrating that a myc-tagged TDP-43 can immunoprecipitate a flag tagged leucine-rich domain expressed in HEK293T cells (FIGS. 6A-6B). It is also the first evidence of its kind to demonstrate that the characteristic NCIs of ALS can be produced in cell culture through a simple metabolic stress and that both TDP-43 and RGNEF participate in this process.

### The Critical Role for RGNEF and the Leucine Rich Domain of RGNEF in Modulating the Toxicity of TDP-43

i. In Vitro Experiments

In order to understand the relationship between TDP-43 and RGNEF, we have developed both an in vitro (cell culture) and in vivo (Drosophila (fruit fly)) model of this interaction. These are the critical experiments that indicate the therapeutic usefulness for full length RGNEF, the leucine rich domain of RGNEF, or a critical interactor with the leucine rich domain in inhibiting the toxicity of TDP-43.

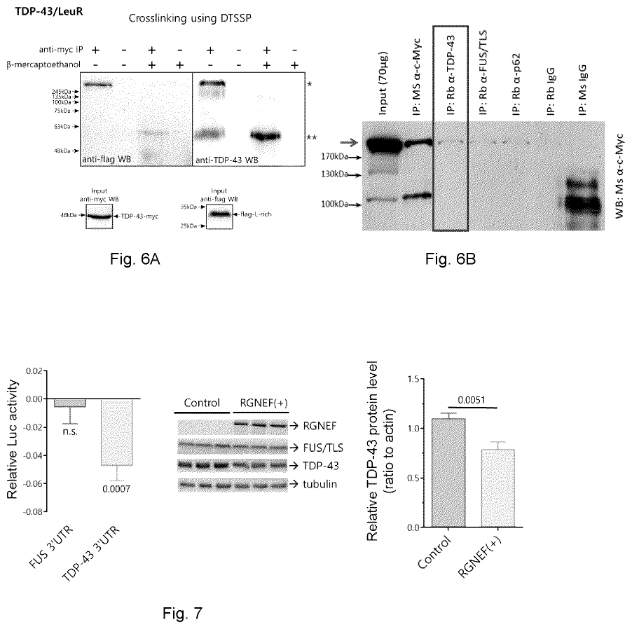

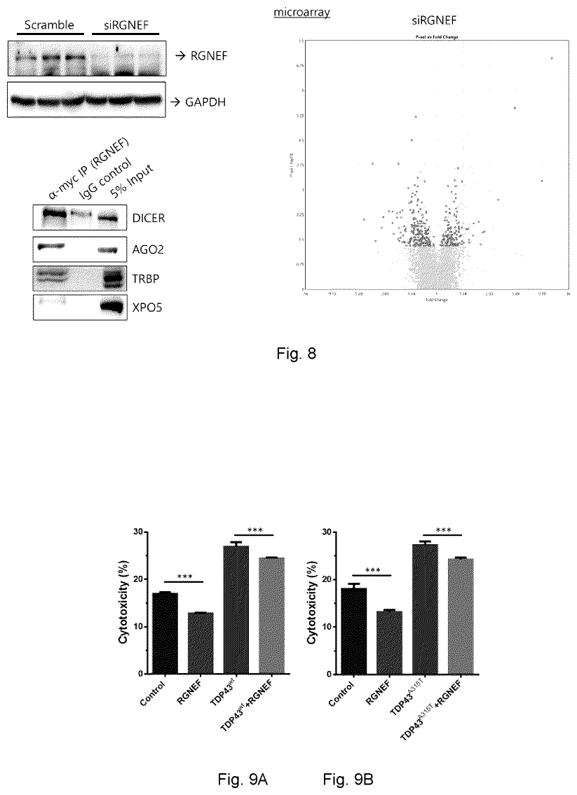

Our evidence that RGNEF is a critical modulator of TDP-43 metabolism is significant. Using a luciferase assay in which the 3′UTR of TDP-43 is linked to a fluorescent reporter which then functions as a marker of RNA stability, we have shown that RGNEF destabilizes TDP-43 3′UTR (FIG. 7). Further, this interaction leads directly to a reduction in TDP-43 protein levels (FIG. 7). The significance of this is that if the over-expression of TDP-43 is core to the pathobiology of ALS, RGNEF is able to suppress this TDP-43 expression. This is further supported by our finding that the suppression of endogenous RGNEF expression using siRNA significantly disrupts miRNA biogenesis, including that of key miRNAs that are regulatory to TDP-43 mRNA stability (FIG. 8). Finally, returning to the cell culture model, full length RGNEF can mitigate the toxicity of TDP-43 (wild type) when both are expressed in HEK293T cells (FIG. 9A). We further extended this observation to a mutant variant of TDP-43 known to be associated with familial ALS (A315T mutation) (FIG. 9B).

ii. In Vivo Experiments

Given the above data supporting a critical role for RGNEF in modulating the toxicity of TDP-43, we next examined the effect of RGNEF and its leucine rich domain in modulating the toxicity of TDP-43 in a drosophila model of motor neuron degeneration in which TDP-43 (human) is expressed. We employed 3 different tissue specific drivers in constructing genetic models: a pan-neuronal driver (Elav), a motor neuron specific driver (D42) and an eye specific driver (GMR).

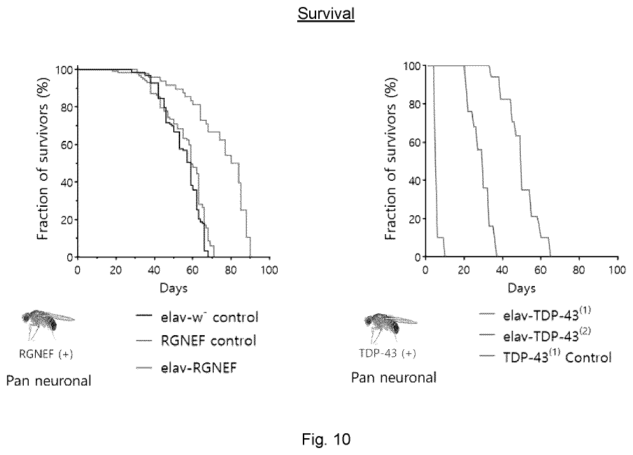

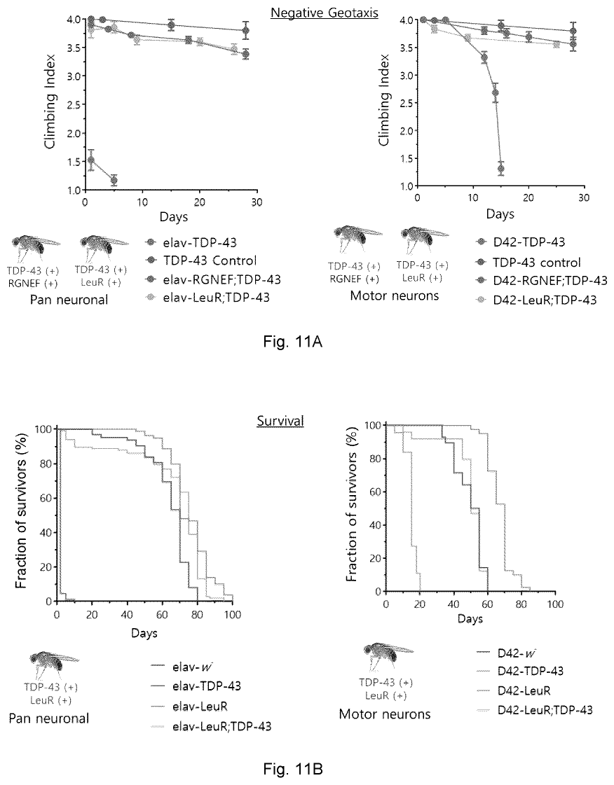

Using the Elav driver for full length RGNEF expression, we observed that the natural lifespan of flies was significantly increased over control flies (FIG. 10). In contrast, and as described in the literature, the expression of wild type TDP-43 in drosophila was associated with a significant reduction in survival (FIG. 10).

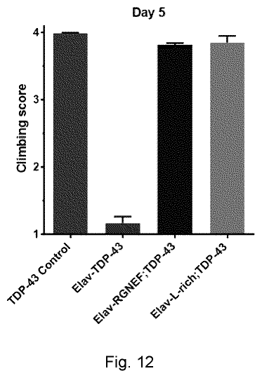

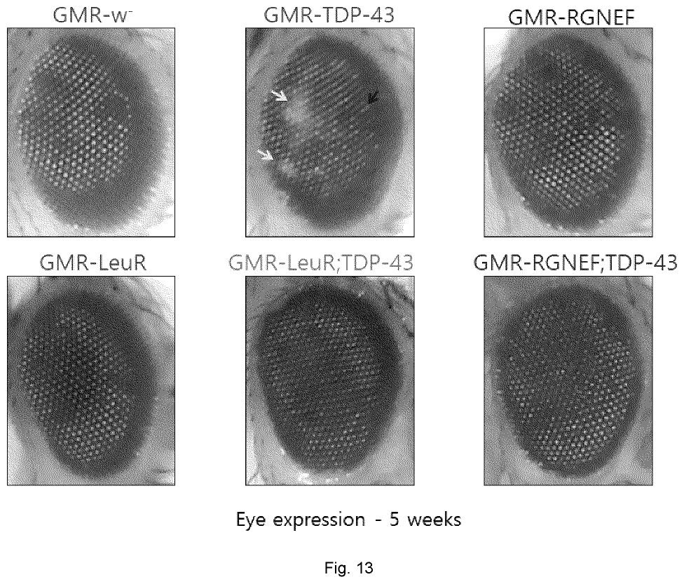

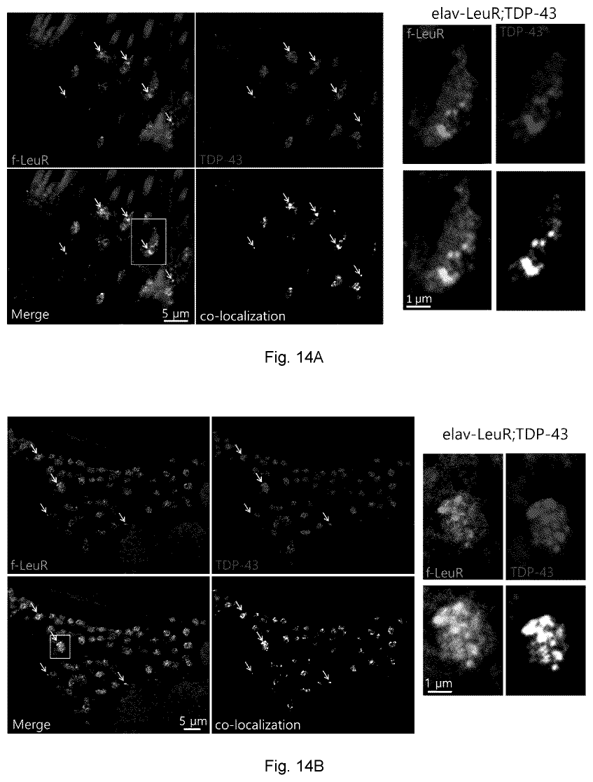

We then extended these studies into double transgenic flies in which we expressed wild type TDP-43 with either full-length RGNEF or a construct expressing only the leucine rich domain of RGNEF. Both survival and negative geotaxis (climbing assay) were used as measures of pathological phenotype. Regardless of whether a pan-neuronal or motor neuron specific driver was utilized in the constructs, a significant improvement in both survival and negative geotaxis was observed (FIGS. 11A-11B). The striking results of the abolishment of the motor dysfunction phenotype in the presence of full length RGNEF or the leucine rich domain of RGNEF is further illustrated in FIG. 12. This observation was further extended to observations of pathology of the outer eye complex in the fly by restricting expression of RGNEF or leucine rich constructs to the eye (FIG. 13). As with survival and motor phenotype, a significant inhibition of the development of eye pathology as driven by wild type TDP-43 was observed. This was accompanied by neuronal cytoplasmic inclusions that demonstrated colocalization of the leucine rich domain of RGNEF with TDP-43 (FIGS. 14A-14B).

Summary. Recall that the most ubiquitous finding in ALS is the massive over-expression of TDP-43 and its' nuclear to cytosolic shift in both sporadic and familial ALS, regardless of the presence or absence of a mutation. Hence, the striking observation that either full-length RGNEF or the leucine-rich region or the leucine-rich domain could substantially or even completely inhibit the TDP-43 effect on the motor function has profound implications for potential therapies in ALS. This suggests that RGNEF recruitment to NCIs, as occurs in ALS, may cause a loss of function effect, thus contributing to neuronal death.

These experiments have demonstrated that RGNEF is a critical RNA binding protein whose metabolism is fundamentally altered in ALS. Further, we have shown that RGNEF, or a fragment of its N-terminal domain, play a critical role in protecting the neuron from metabolic injury, and critical to the pathogenesis of ALS, can fully inhibit the toxicity of over-expressed TDP-43 which is the hallmark of the disorder.

### Example 2—PH Domain of RGNEF Regulates its Nuclear-Cytoplasmic Localization

### Materials and Methods

### 1. Constructs

The plasmid encoding human RGNEF (pcDNA-RGNEF-myc) was previously generated in our lab (Droppelmann et al., 2013). pcDNARGNEF-ΔPH-myc, a deletion construct lacking the PH domain (residues 1083-1201) was generated by standard molecular biology procedures. Site-directed mutagenesis (QuickChange Lightning Multi Site-Directed Mutagenesis Kit; Agilent Technologies) of pcDNA-RGNEF-myc was used to mutate basic residues of the NLS to neutral alanine residues (R1101 A, K1103 A, K1120 A, & K1123 A) and create a myc-tagged NLSlacking construct pcDNA-RGNEF-mutNLS-myc.

The PH domain of either wild-type RGNEF or of our mutant NLS RGNEF were inserted into either pHM830 or pHM840 vectors (Addgene) resulting in the expression of fusion proteins of 160 kDa.

### 2. Cell Lines and Transfections

HEK293 T and SH-SY5Y cells were maintained in Dulbecco's modified Eagle's medium (Life Technologies) supplemented with 10% fetal bovine serum (Life Technologies). SH-SY5Y cells were treated with 10 μM retinoic acid (Sigma-Aldrich) for 3 days prior to transfection to induce a neuron-like phenotype (Encinas et al., 2000). Transient transfections were performed using Lipofectamine 2000 (Life Technologies) in 6-well plates according to the manufacturer's protocols.

### 3. Subcellular Fractionation

Nuclear and cytoplasmic fractions were isolated as previously described (Liu and Fagotto, 2011). Briefly, cell membranes were semipermeabilized by 10 min incubation in 42 μg/ml digitonin (Sigma-Aldrich) dissolved in 1×NEH buffer (150 mM NaCl, 0.2 mM EDTA, 20 mM Hepes-NaOH—pH 7.4). The solution was then collected and stored as “cytoplasmic fraction”. Plates were scraped, and the remaining cellular components were collected and homogenized using a Dounce homogenizer. The sample was centrifuged at 1,000×g for 10 min and the supernatant was collected as “nuclear fraction”.

### 4. Western Blot

Protein samples were mixed with loading buffer containing SDS and separated electrophoretically by 10% SDS-PAGE. Proteins were transferred to nitrocellulose membranes and then incubated with primary antibodies for 90 min and after with secondary antibodies for 60 min. Immunoblots were developed using chemiluminescence (Western Lightning Plus ECL; PerkinElmer) and visualized with a Bio-Rad ChemiDoc XRS+system. Primary antibodies were directed against cmyc (1:4000; Cedarlane; cat #CLX229AP), GAPDH (1:2000; Abcam; cat #ab9485), and Lamin A/C (1:500; Santo Cruz; cat #ab68417). Secondary antibodies were HRP conjugated: Goat anti-mouse (1:5000; Bio-Rad; cat #170-6516), Swine anti-rabbit (1:2500; Dako; cat #P039901-2), and Mouse anti-rabbit (1:5000; Santa Cruz; cat #1721011).

### 5. GEF Activity

The activation of RhoA by RGNEF in HEK293T cells was determined using the “G-LISA Rho Activation Assay Biochem Kit (Luminescence based)” (Cytoskeleton Inc.) according to the manufacturer's instructions. Cells were transfected with pcDNA-RGNEF-myc, pcDNA-RGNEFmutNLS-myc, and pcDNA3.1-myc-His as control. The activation of RhoA, dependant on RGNEF's GEF activity, was evaluated in basal conditions after 6 h of serum starving.

### 6. Immunofluorescence and Confocal Microscopy

Briefly, coverslips were fixed by incubating for 15 min in 4% paraformaldehyde solution and permeabilized by incubating for 10 min in 0.2% Triton X-100. Coverslips were then incubated in 50 mM ammonium chloride for 30 min to quench aldehyde groups and reduce background staining. Coverslips were blocked with 8% bovine serum albumin (BSA; Fisher Scientific Company) and then incubated with primary antibody for 90 min at room temperature and then incubated for 60 min with secondary antibody. Primary antibodies directed against c-myc (1:250, Cedarlane; cat #CLX229AP) and R III Tubulin (1:60, Sigma-Aldrich; cat #T8578). Secondary antibodies goat antimouse Alexa Fluor 488 (1:800, Life Technologies; cat #A11029), and goat anti-rabbit Alexa Fluor 546 (1:800, Life Technologies; cat #A11035) were used. The visualization of the actin cytoskeleton in SHSY5Y cells was performed in basal conditions after 6 h of serum starving using the “F-actin visualization biochem kit” (Cytoskeleton Inc.). SHSY5Y cells were used because they form better actin fibers (f-actin) compared to HEK293T cells. Cover slips were incubated in 1 μg/mL Hoechst stain to visualize nuclei. Cover slips were mounted to microscope slides using fluorescent mounting media (Dako). All samples were examined using a Confocal Laser Scanning Platform Microscope (Leica SP8 or Olympus Fluorview 1000 microscopes) and LAS X software (Leica Microsystems Inc.) or FV10-ASW Software (Olympus Corp.).

The percentage of cells showing expression in the nucleus was calculated by counting the number of cells showing nuclear localization and dividing by the total cells expressing the protein (n=100). Nuclear:cytoplasmic ratios were calculated using intensity profiles generated by LAS X software. The mean intensity within either the nuclear or cytoplasmic compartments was determined and expressed as a ratio.

### 7. Statistical Analysis

All statistical analyses were done using GraphPad Prism 6. One-way ANOVA with Tukey post-hoc test was performed to determine statistical significance and obtain p values.

### 8. In Silico Analysis

The online service cNLS mapper (http://nls-mapper.iab.keio.ac.jp/cgi-bin/NLS_Mapper_form.cgi) was used to predict the location of putative NLSs. The software predicts NLSs based on the results of a series of activity-based profiles using synthetic NLSs of different affinities (Kosugi et al., 2008a, a; Kosugi et al., 2009b). Molecular modeling was performed using I-Tasser software (http://zhanglab.ccmb.med.umich.edu/I-TASSER/), which predicts protein structure using modeling by iterative threading assembly simulation (Roy et al., 2010; Yang et al., 2015; Zhang, 2008). The models were then visualized using RasTop 2.2 (by Philippe Valadon). We used the online service NetNES 1.1 (http://www.cbs.dtu.dk/services/NetNES/), which identifies putative NES using a machine learning prediction to assess NES scores to residues (la Cour et al., 2004). We used the online service Clustal Omega (http://www.ebi.ac.uk/Tools/msa/clustalo/)—which aligns proteins based on sequence identity—to identify the specific residues responsible for NES activity.

### Results

### 1. RGNEF Contains a PH Domain-Embedded NLS

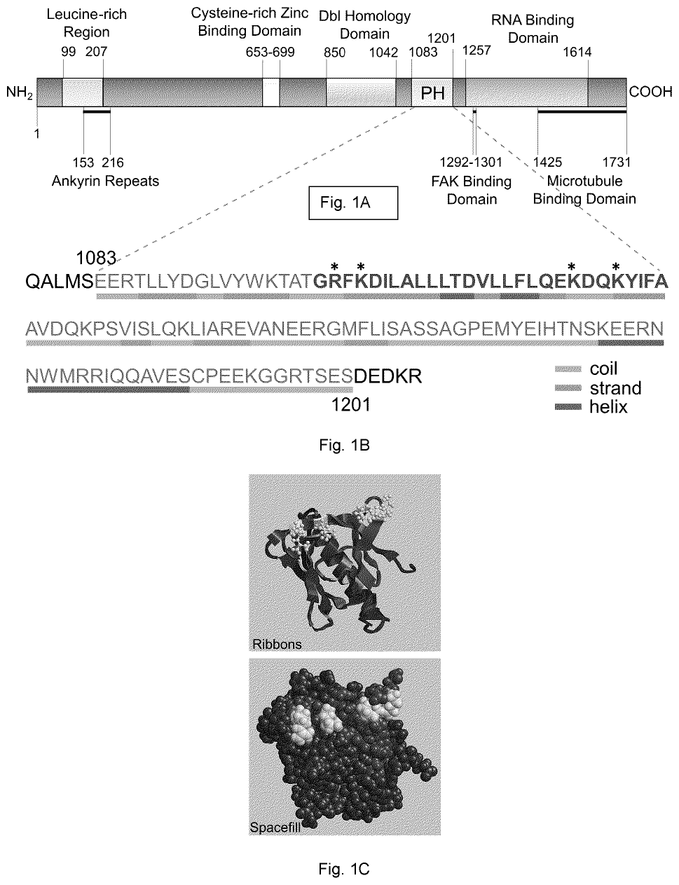

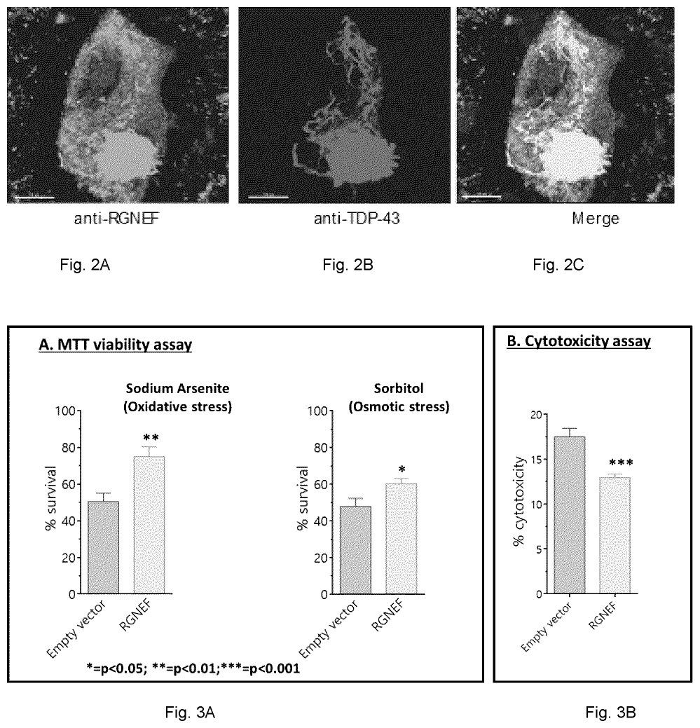

RGNEF contains a Pleckstrin Homology (PH) domain and a Dbl Homology (DH) domain (Tavolieri et al., 2019) (FIG. 1A) in the carboxy terminal half of the protein, which are responsible for RGNEF's GEF activity. In addition to a conserved sequence motif, PH domains are characterized by their distinctive folding: a seven-stranded anti-parallel β-sandwich that is closed at one end by a C-terminal alpha helix (Cozier et al., 2004; Lemmon, 2004). We used I-Tasser software (Roy et al., 2010; Yang et al., 2015) to predict the folding structure of the PH domain of RGNEF from its amino acid sequence. This yielded a seven-stranded anti-parallel β-sandwich with a C-terminal alpha helix consistent with the consensus structure expected for PH domains (FIGS. 1B and 1C). To determine whether the PH domain of RGNEF contained a functional nuclear localization signal (NLS), we analyzed the amino acid sequence of the PH domain using the cNLS Mapper software (Kosugi et al., 2008a; Kosugi et al., 2009a; Kosugi et al., 2009b). This software predicted a bipartite NLS from residues 1100-1127 of the PH domain (FIG. 1B; SEQ ID NO:13) with the basic residues, expected to interact with importin-α, located either outside of, or towards the edges of, the β-sheets where they would localize to the exterior of the protein and likely be accessible for interaction (FIG. 1C). These data suggest that this region could possibly serve as a functional NLS.

### 2. The PH Domain-Embedded NLS is Necessary for RGNEF Nuclear Localization

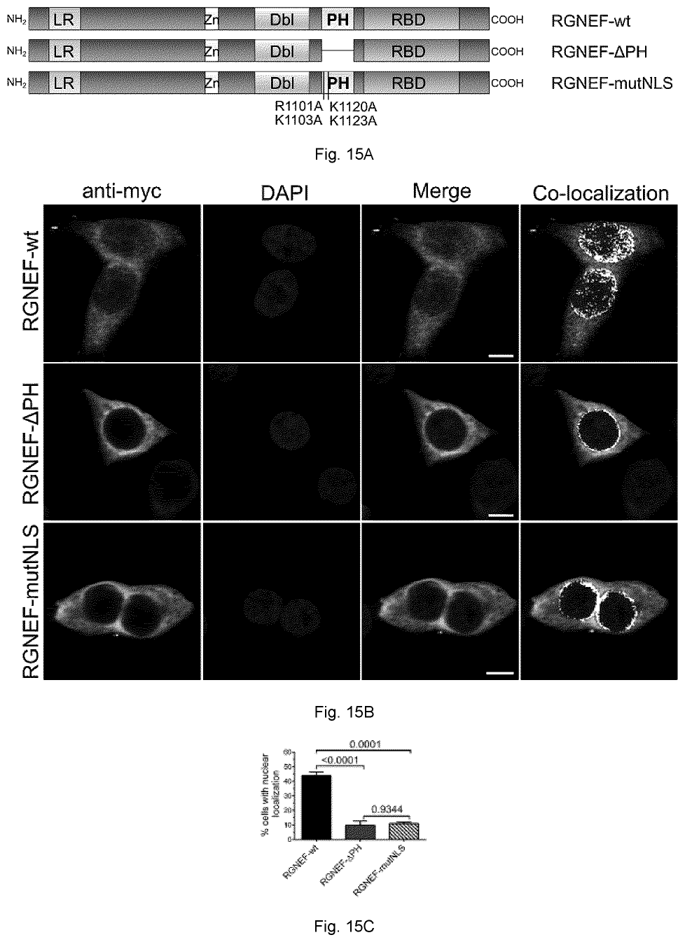

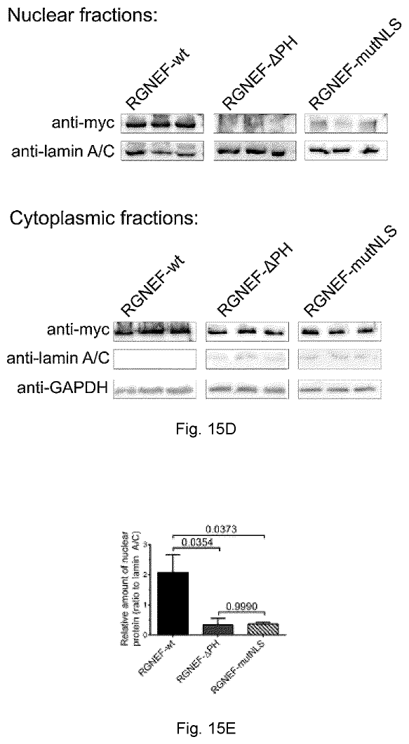

To analyze the functionality of this putative NLS signal, we created two mutant constructs of RGNEF: pcDNA-RGNEF-ΔPH-myc which included a deletion of the 119 residues of the PH domain (del 1084-1202) and pcDNA-RGNEF-mutNLS-myc in which four basic residues of the putative NLS were point-mutated to neutral alanine residues (R1101 A, K1103 A, K1120 A, and K1123 A) without inducing major changes in the predicted folding of the protein (FIG. 15A). HEK293 T cells transfected with the vector expressing wild-type (wt) RGNEF (pcDNA-RGNEF-myc) showed predominantly cytoplasmic localization of the protein with moderate levels of nuclear localization (FIG. 15B), consistent with what we observed previously (Droppelmann et al., 2013). Both NLS lacking constructs showed protein largely absent from the nucleus (FIG. 15B).

To quantify the difference in cellular localization between constructs, we analyzed the distribution of the proteins in transfected cells using intensity profiles generated by LAS X software and determined the percentage of construct expressing cells that showed protein localized to the nucleus. Cells transfected with either mutant construct had a significantly lower percentage of cells showing nuclear localization than those expressing RGNEF-wt (RGNEF-ΔPH, p<0.0001; RGNEF-mutNLS p=0.0001) (FIG. 15C). We further quantified differences in nuclear localization using subcellular fractionation and western blot. A statistically significant difference was observed in nuclear fractions in which cells expressing RGNEF-wt show significantly greater nuclear levels of protein than observed for either of the two mutants (RGNEF-ΔPH, p=0.0354; RGNEF-mutNLS p=0.0373, FIGS. 15D and 15E) in which the NLS was deleted or pointmutated.

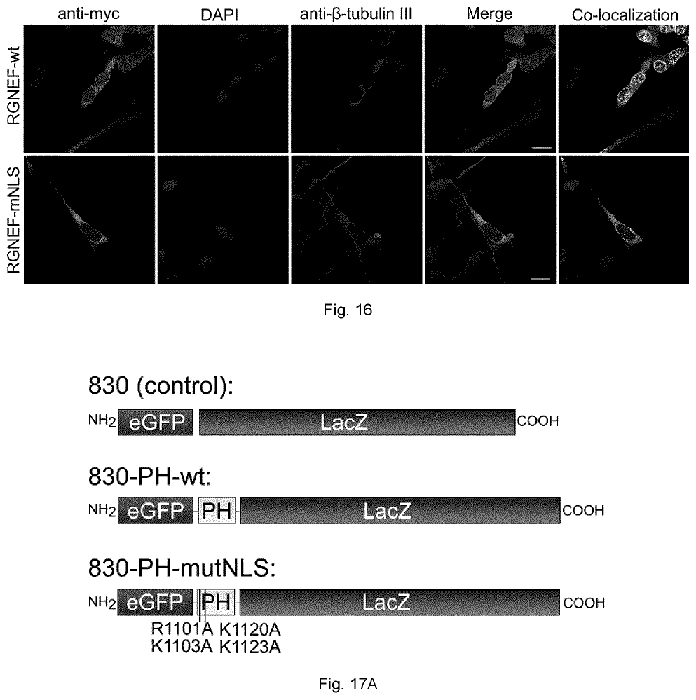

We confirmed the role of the putative NLS in SH-SY5Y cells. This neuroblastoma-derived cell line can be differentiated to yield a neuronlike, non-proliferating phenotype using retinoic acid (Encinas et al., 2000). Consistent with the observations using HEK293T cells, differentiated SH-SY5Y cells showed a mixed nuclear/cytoplasmic distribution of RGNEF-wt, whereas cells expressing RGNEF-mutNLS had RGNEF mostly excluded from the nucleus (FIG. 16).

From the functional point of view, RGNEF carrying the mutated NLS was unable to activate RhoA in contrast RGNEF-wt in which RhoA activation was observed. Additionally, RGNEF-mutNLS was unable to induce the formation of f-actin when overexpressed, in contrast to that observed with RGNEF-wt transfections.

These results suggest that the PH domain of RGNEF contains a putative NLS and confirms that the 4 basic residues are critical to mediate nuclear localization and in addition, contribute directly to RGNEF's GEF activity.

3. The PH Domain of RGNEF Alone is Sufficient to Translocate a 160 kDa Protein into the Nucleus

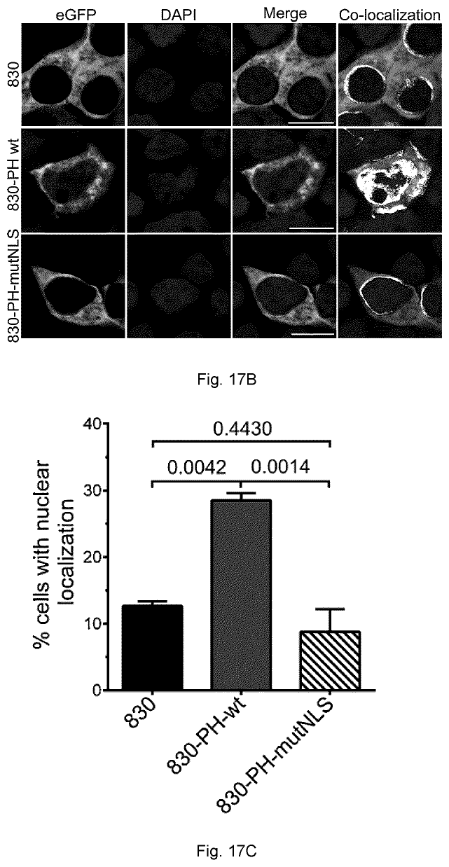

Having observed that the NLS was necessary for the nuclear localization of full length RGNEF, we next determined whether this domain alone was sufficient for the nuclear localization of a protein greater than 110 kDa, the maximum proposed threshold for passive diffusion of protein to the nucleus (Wang and Brattain, 2007). For this purpose, we utilized the pHM830 vector (Sorg and Stamminger, 1999) which adds an N-terminal green fluorescent protein (eGFP) and a C-terminal betagalactosidase (LacZ) to the insert of interest in the multicloning site. Plasmids pHM830-PH-wt and pHM830-PH-mutNLS generated the 160 kDa fusion proteins: 830-PH-wt and 830-PH-mutNLS (FIG. 17A). When expressed in HEK293T cells (FIG. 17B), 830-PH-wt showed moderate levels of nuclear localization consistent with the observed with RGNEF-wt expression (FIG. 15B). Cells expressing the point-mutated form of the PH domain, 830-PH-mutNLS, showed a reduced amount of nuclear localization compared to 830-PH-wt, similar to the amount observed in cells transfected with pHM830 vector alone as control of cytoplasmic localization (FIG. 17B). Quantification of cells expressing the proteins found that a significantly higher percentage of cells containing 830-PHwt showed nuclear localization than either 830-PH-mutNLS (p=0.0014) or control (empty vector) (p=0.0042) (FIG. 17C). These results suggest that the presence of the PH domain is sufficient to translocate a protein greater than the largest passively diffusible size described (110 kDa) into the nucleus.

### 4. The PH Domain-Embedded NLS Contains an Overlapping NES

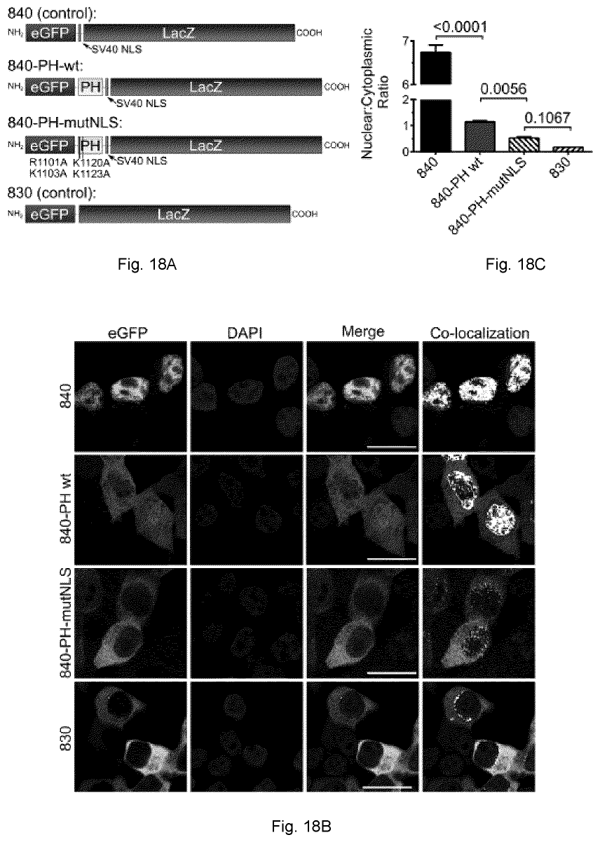

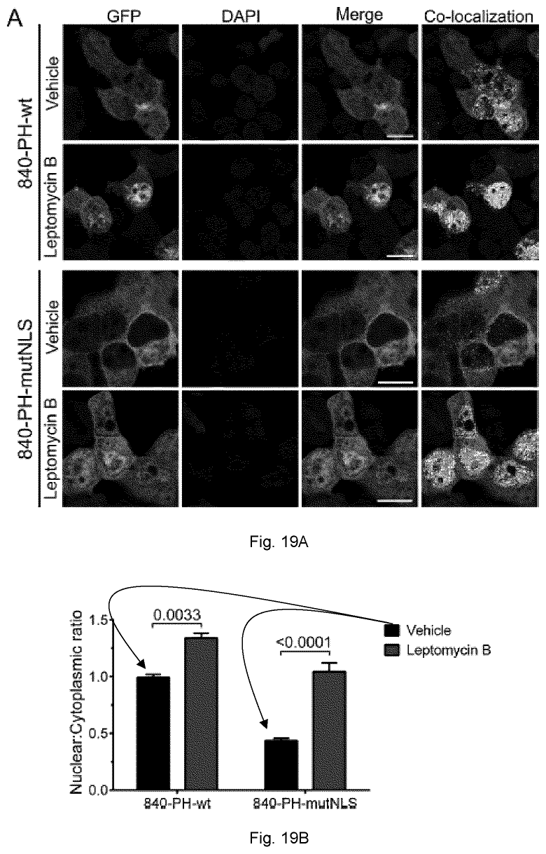

Interestingly, 830-PH-wt showed moderate levels of cytoplasmic localization, even though we would expect localization to be predominantly nuclear in an NLS containing protein. This suggested that the PH domain may also contains a NES. For this reason, we utilized the pHM840 vector (Sorg and Stamminger, 1999), which like the pHM830 vector encodes for the addition of both eGFP and LacZ, but also contains the SV40 NLS for nuclear localization (FIG. 18A). When expressed in HEK293 T cells, the protein encoded by the pHM840 vector alone localized almost exclusively to the nucleus as expected for a large, NLS containing protein (FIG. 18B). Quantified as nuclear:cytoplasmic ratio to obtain a measurement of the degree of translocation of the different constructs, nuclear localization was significantly decreased in cells expressing 840-PH-wt, suggesting that the PH domain contains a sequence sufficient to induce nuclear export despite the presence of the two NLS signals (p<0.0001). Cells containing 840-PH-mutNLS showed even lower levels of nuclear localization than 840-PH-wt (p=0.0056), consistent with what we would expect given that it contains the inactivated mutant NLS of the PH domain, but still having a functional SV40 NLS. As expected, the protein encoded by the PHM830 vector only shows cytoplasmic localization (FIG. 18C). These data suggests the presence of an active NES in the PH domain of RGNEF and also support our previous finding that the wild-type PH contains an active NLS.

We then used in silico techniques to identify the location of the NES. The software NetNES 1.1 (la Cour et al., 2004) yielded NES scores above threshold for 9 residues of a 10 residue stretch within the linker region of the bipartite NLS, suggesting the presence of an NES within this region. Alignment against the amino acid sequences of nine other NES previously identified in the literature (Kutay and Guttinger, 2005) found conserved identity among the hydrophobic residues of the traditional consensus NES motif Φ1X(2,3)Φ2X(2,3)Φ3XΦ4 ((n=L, V, I, F, or M) (Bogerd et al., 1996) within this region. Comparison of this region with the six subclasses of NES sequences (Kosugi et al., 2008b) showed that, because of the high concentration of hydrophobic residues, this region can constitute any of the six subclasses of NES described. Molecular modeling of the PH domain shows that, although when observed as a linear amino acid sequence the NES lies within the NLS, when depicted as a molecular model the residues of the NLS and NES are in close proximity but independent of one another.

### 5. Nuclear Export of the PH Domain is Exportin-1 Dependent

To confirm the presence of a classical NES embedded within the PH domain, we treated HEK293 T cells with Leptomycin-B (LMB) for 4 h, 20 h after the transfection with either of the two pHM840 constructs expressing 840-PH-wt or 840-PH-mutNLS. LMB specifically blocks NES dependent nuclear export by covalently binding exportin-1 (Kudo et al., 1999). Cells expressing either construct of the PH domain showed significantly higher levels of nuclear localization (840-PH-wt, p=0.0033; 840-PH-mutNLS, p<0.0001) when treated with LMB than when treated with vehicle (FIGS. 19A and 19B), suggesting that this region does function as a NES, and that it functions in an exportin-1—dependent manner.

Sequence Listings

a) Human RGNEF (SEQ ID NO: 13):
MELSCSEAPLYGQMMIYAKFDKNVYLPEDAEFYFTYDGSHQRHVMIAERIEDNV
LQSSVPGHGLQETVTVSVCLCSEGYSPVTMGSGSVTYVDNMACRLARLLVTQAN
RLTACSHQTLLTPFALTAGALPALDEELVLALTHLELPLEWTVLGSSSLEVSSHRE
SLLHLAMRWGLAKLSQFFLCLPGGVQALALPNEEGATPLDLALREGHSKLVEDV
TNFQGRWSPSFSRVQLSEEASLHYIHSSETLTLTLNHTAEHLLEADIKLFRKYFWD
RAFLVKAFEQEARPEERTAMPSSGAETEEEIKNSVSSRSAAEKEDIKRVKSLVVQH
NEHEDQHSLDLDRSFDILKKSKPPSTLLAAGRLSDMLNGGDEVYANCMVIDQVG
DLDISYINIEGITATTSPESRGCTLWPQSSKHTLPTETSPSVYPLSENVEGTAHTEAQ
QSFMSPSSSCASNLNLSFGWHGFEKEQSHLKKRSSSLDALDADSEGEGHSEPSHIC
YTPGSQSSSRTGIPSGDELDSFETNTEPDFNISRAESLPLSSNLQSKESLLSGVRSRS
YSCSSPKISLGKTRLVRELTVCSSSEEQRAYSLSEPPRENRIQEEEWDKYIIPAKSES
EKYKVSRTFSFLMNRMTSPRNKSKTKSKDAKDKEKLNRHQFAPGTFSGVLQCLV
CDKTLLGKESLQCSNCNANVHKGCKDAAPACTKKFQEKYNKNKPQTILGNSSFR
DIPQPGLSLHPSSSVPVGLPTGRRETVGQVHPLSRSVPGTTLESFRRSATSLESESD
HNSCRSRSHSDELLQSMGSSPSTESFIMEDVVDSSLWSDLSSDAQEFEAESWSLVV
DPSFCNRQEKDVIKRQDVIFELMQTEMHHIQTLFIMSEIFRKGMKEELQLDHSTVD
KIFPCLDELLEIHRHFFYSMKERRQESCAGSDRNFVIDRIGDILVQQFSEENASKM
KKIYGEFCCHHKEAVNLFKELQQNKKFQNFIKLRNSNLLARRRGIPECILLVTQRI
TKYPVLVERILQYTKERTEEHKDLRKALCLIKDMIATVDLKVNEYEKNQKWLEIL
NKIENKTYTKLKNGHVFRKQALMSEERTLLYDGLVYWKTATGRFKDILALLLTD
VLLFLQEKDQKYIFAAVDQKPSVISLQKLIAREVANEERGMFLISASSAGPEMYEI
HTNSKEERNNWMRRIQQAVESCPEEKGGRTSESDEDKRKAEARVAKIQQCQEIL
TNQDQQICAYLEEKLHIYAELGELSGFEDVHLEPHLLIKPDPGEPPQAASLLAAAL
KEAESLQVAVKASQMGAVSQSCEDSCGDSVLADTLSSHDVPGSPTASLVTGGRE
GRGCSDVDPGIQGVVTDLAVSDAGEKVECRNFPGSSQSEIIQAIQNLTRLLYSLQA
ALTIQDSHIEIHRLVLQQQEGLSLGHSILRGGPLQDQKSRDADRQHEELANVHQL
QHQLQQEQRRWLRRCEQQQRAQATRESWLQERERECQSQEELLLRSRGELDLQL
QEYQHSLERLREGQRLVEREQARMRAQQSLLGHWKHGRQRSLPAVLLPGGPEV
MELNRSESLCHENSFFINEALVQMSFNTFNKLNPSVIHQDATYPTTQSHSDLVRTS
EHQVDLKVDPSQPSNVSHKLWTAAGSGHQILPFHESSKDSCKNGSSMTKCSCTLT
SPPGLWTGTTSTLKDLDTSHTESPTPHDSNSHRPQLQAFITEAKLNLPTRTMTRQD
GETGDGAKENIVYL
Code:
From top to bottom:
First underline: Leu-rich domain
Second underline: Zn binding domain
Third underline: DH domain
Fourth underline: PH domain
Fifth underline: RNA binding domain
b) PH Domain (SEQ ID NO: 14)
EERTLLYDGLVYWKTATGRFKDILALLLTDVLLFLQEKDQKYIFAAVDQKPSVIS
LQKLIAREVANEERGMFLISASSAGPEMYEIHTNSKEERNNWMRRIQQAVESCPE
EKGGRTSES
c) Region of RGNEF that contains the Leu-rich domain
DNA sequence (SEQ ID NO: 15):
atggagttgagctgcagcgaagcacctctttacgggcagatgatgatctatgcgaagtttgacaaaaatgtgtatcttcctgaagat
gctgagttttactttacttatgacggatctcatcagcgacatgtcatgattgcagagcgcatcgaggataacgttctccagtccagcg
tcccaggccatgggcttcaggagacggtgacggtatctgtgtgcctctgctcggaaggttactctccggtgaccatgggctctgg
ctcagtgacctacgtggacaacatggcttgcaggctggctcgtctgctggtgacgcaggccaatcgcctcacagcctgcagcca
ccagaccctgctgaccccatttgccttgacggcaggagcactgcctgccttggatgaggagctcgtgctggctctgacccatctg
gaattgcctctagagtggactgtgttgggaagttcttcacttgaagtatcttctcacagagaatctcttctacacctggctatgagatg
gggcctggctaaactttcccagttcttcttgtgtctcccggggggagtccaggccttggctttacccaacgaagagggtgccacac
cattagacttagctttacgtgaaggacactccaagctggtggaagacgtcacaaattttcagggcagatggtccccaagcttctcc
cgagtgcagctcagtgaagaagcctccttgcattac
Protein sequence (SEQ ID NO: 16):
MELSCSEAPLYGQMMIYAKFDKNVYLPEDAEFYFTYDGSHQRHVMIAERIEDNV
LQSSVPGHGLQETVTVSVCLCSEGYSPVTMGSGSVTYVDNMACRLARLLVTQAN
RLTACSHQTLLTPFALTAGALPALDEELVLALTHLELPLEWTVLGSSSLEVSSHRE
SLLHLAMRWGLAKLSQFFLCLPGGVQALALPNEEGATPLDLALREGHSKLVEDV
TNFQGRWSPSFSRVQLSEEASLHY

### REFERENCES

Bogerd, H. P., R. A. Fridell, R. E. Benson, J. Hua, and B. R. Cullen. 1996. Protein sequence requirements for function of the human T-cell leukemia virus type 1 Rex nuclear export signal delineated by a novel in vivo randomization-selection assay. Mol Cell Biol. 16:4207-4214.
Cheung, K., C. A. Droppelmann, A. MacLellan, I. Cameron, B. Withers, D. Campos-Melo, K. Volkening, and M. J. Strong. 2017. Rho guanine nucleotide exchange factor (RGNEF) is a prosurvival factor under stress conditions. Mol Cell Neurosci. 82:88-95.
Cozier, G. E., J. Carlton, D. Bouyoucef, and P. J. Cullen. 2004. Membrane targeting by pleckstrin homology domains. Curr Top MicrobiolImmunol. 282:49-88.
Droppelmann, C. A., D. Campos-Melo, M. Ishtiaq, K. Volkening, and M. J. Strong. 2014. RNA metabolism in ALS: when normal processes become pathological. Amyotroph Lateral Scler Frontotemporal Degener. 15:321-336.
Droppelmann, C. A., B. A. Keller, D. Campos-Melo, K. Volkening, and M. J. Strong. 2013. Rho guanine nucleotide exchange factor is an NFL mRNA destabilizing factor that forms cytoplasmic inclusions in amyotrophic lateral sclerosis. Neurobiol Aging. 34:248-262.
Encinas, M., M. Iglesias, Y. Liu, H. Wang, A. Muhaisen, V. Cena, C. Gallego, and J. X. Comella. 2000. Sequential treatment of SH-SY5Y cells with retinoic acid and brain-derived neurotrophic factor gives rise to fully differentiated, neurotrophic factor-dependent, human neuron-like cells. J Neurochem. 75:991-1003.
Keller, B. A., K. Volkening, C. A. Droppelmann, L. C. Ang, R. Rademakers, and M. J. Strong. 2012. Co-aggregation of RNA binding proteins in ALS spinal motor neurons: evidence of a common pathogenic mechanism. Acta Neuropathol. 124:733-747.
Kosugi, S., M. Hasebe, T. Entani, S. Takayama, M. Tomita, and H. Yanagawa. 2008a. Design of peptide inhibitors for the importin alpha/beta nuclear import pathway by activity-based profiling. Chem Biol. 15:940-949.
Kosugi, S., M. Hasebe, N. Matsumura, H. Takashima, E. Miyamoto-Sato, M. Tomita, and H. Yanagawa. 2009a. Six classes of nuclear localization signals specific to different binding grooves of importin alpha. J Biol Chem. 284:478-485.
Kosugi, S., M. Hasebe, M. Tomita, and H. Yanagawa. 2008b. Nuclear export signal consensus sequences defined using a localization-based yeast selection system. Traffic. 9:2053-2062.
Kosugi, S., M. Hasebe, M. Tomita, and H. Yanagawa. 2009b. Systematic identification of cell cycle-dependent yeast nucleocytoplasmic shuttling proteins by prediction of composite motifs. Proc Natl Acad Sci USA. 106:10171-10176.
Kutay, U., and S. Guttinger. 2005. Leucine-rich nuclear-export signals: bom to be weak. Trends Cell Biol. 15:121-124.
la Cour, T., L. Kiemer, A. Molgaard, R. Gupta, K. Skriver, and S. Brunak. 2004. Analysis and prediction of leucine-rich nuclear export signals. Protein Eng Des Sel. 17:527-536.
Lemmon, M. A. 2004. Pleckstrin homology domains: not just for phosphoinositides. Biochem Soc Trans. 32:707-711.
Roy, A., A. Kucukural, and Y. Zhang. 2010. I-TASSER: a unified platform for automated protein structure and function prediction. Nat Protoc. 5:725-738.
Sorg, G., and T. Stamminger. 1999. Mapping of nuclear localization signals by simultaneous fusion to green fluorescent protein and to beta-galactosidase. Biotechniques. 26:858-862.
Tavolieri, M. V., C. A. Droppelmann, D. Campos-Melo, K. Volkening, and M. J. Strong. 2019. A novel overlapping NLS/NES region within the PH domain of Rho Guanine Nucleotide Exchange Factor (RGNEF) regulates its nuclear-cytoplasmic localization. Eur J Cell Biol. 98:27-35.
Volkening, K., C. Leystra-Lantz, and M. J. Strong. 2010. Human low molecular weight neurofilament (NFL) mRNA interacts with a predicted p190RhoGEF homologue (RGNEF) in humans. Amyotroph Lateral Scler. 11:97-103.
Wang, R., and M. G. Brattain. 2007. The maximal size of protein to diffuse through the nuclear pore is larger than 60 kDa. FEBS Lett. 581:3164-3170.
Wu, J., J. Zhai, H. Lin, Z. Nie, W. W. Ge, L. Garcia-Bermejo, R. J. Muschel, W. W. Schlaepfer, and R. Canete-Soler. 2003. Cytoplasmic retention sites in p190RhoGEF confer anti-apoptotic activity to an EGFP-tagged protein. Brain Res Mol Brain Res. 117:27-38.
Yang, J., R. Yan, A. Roy, D. Xu, J. Poisson, and Y. Zhang. 2015. The I-TASSER Suite: protein structure and function prediction. Nat Methods. 12:7-8.
Yu, H. G., J. O. Nam, N. L. Miller, I. Tanjoni, C. Walsh, L. Shi, L. Kim, X. L. Chen, A. Tomar, S. T. Lim, and D. D. Schlaepfer. 2011. p190RhoGEF (Rgnef) promotes colon carcinoma tumor progression via interaction with focal adhesion kinase. Cancer Res. 71:360-370.

### Example 3—RGNEF Depletion in Neuronal Cells Alters the Expression of 1607 Genes

### Material and Methods

Total RNA was extracted from RNA interference negative control and RGNEF depleted SH-SY5Y cells using miRNeasy Kit (Qiagen). Library construction and RNA sequencing was performed by Novogene (https://en.novogene.com/) on three independent experiments obtained for each tested sample.

Before proceeding to the cDNA library construction and transcriptome analysis, all the samples were tested for:
1) RNA purity using Nanodrop ((OD260/OD280);
2) RNA degradation and contamination using agarose gel electrophoresis;
3) RNA integrity using Agilent 2100.

Then, in order to create the cDNA library, mRNA was enriched using oligo(dT) beads and randomly fragmented. Reverse transcription with random hexamers were used in order to obtain cDNA. After first-strand synthesis, second strand was produced using a custom second-strand synthesis buffer (Illumina), dNTPs, RNase H and Escherichia coli polymerase I. The final cDNA library was ready after a round of purification, terminal repair, A-tailing, ligation of sequencing adapters, size selection and PCR enrichment. Library concentration and insert size were then estimated using Qubit 2.0 fluorometer (Life Technologies) and quantitative PCR (Q-PCR), respectively. RNA-seq analysis were performed using Illumina HiSeq NovaSeq 600 instrument. The original raw data from Illumina were transformed to sequenced reads by CASAVA base recognition (base calling). Raw Data was stored in FASTQ(fq) format files, which contain reads sequence and corresponding base quality. GC content distribution was evaluated to detect potential AT/GC separation. Low quality reads and reads containing adapter were removed from the analysis. Therefore, raw reads were filtered as follows:
Discard reads with adapter contamination.
Discard reads when uncertain nucleotides constitute more than 10 percents of either read (N>10%).
Discard reads when low quality nucleotides (base quality less than 20) constitute more than 50 percents of the read.

Then, clean reads were mapped to the reference genome using STAR software and the mapping files were provided in BAM format.

HTSeq software was used to analyze the gene expression levels, using the union mode. To compare the gene expression levels between different genes and experiments, readcounts were normalized for gene length and sequencing depth and FPKM were generated. In general, an FPKM value of 0.1 or 1 is set as the threshold for determining whether the gene is expressed or not.

Pearson correlation (R2) among biological replicates was also tested.

Differential gene expression analysis was carried out using DEseq2 R package, following these steps:
1) Readcounts Normalization;
2) Model dependent p-value estimation;
3) FDR value estimation based on multiple hypothesis testing (Benjamini and Hochberg's approach).

The overall distribution of differentially expressed genes (DEGs) were evaluated using the following cut-off: up-regulated genes Fold Change>1.3 and padjust<0.05; down-regulated genes Fold Change <0.7 and padjust<0.05.

GOseq package from R (Young et al., 2010) was also used for KEGG pathway analysis of RNA-seq data. Categories significantly enriched (padjust<0.05) were considered.

### Results.

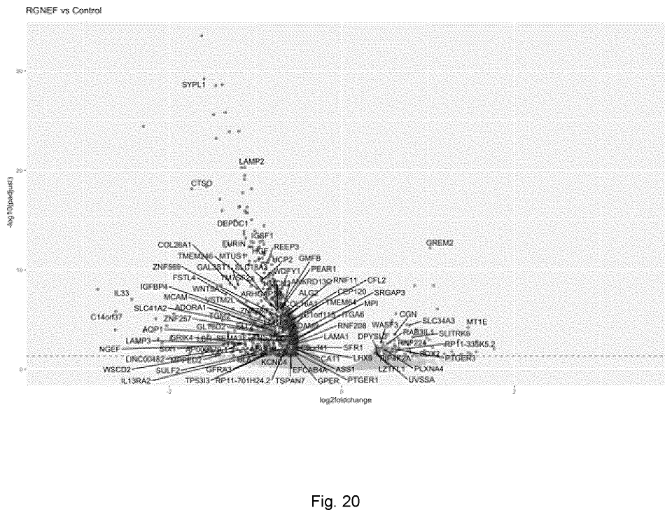

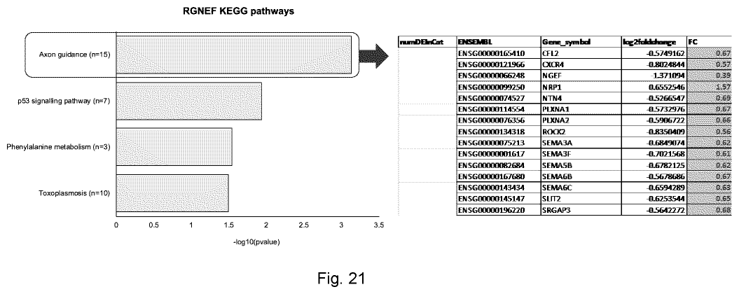

The RNAseq analysis showed that 1607 genes are significantly altered in SH-SY5Y cells when RGNEF is depleted. From those genes 319 were up-regulated and 1288 were down-regulated (Table 4 and Table 5, FIG. 20). The KEGG pathway analysis shows that the expression of several genes related to axon guidance are altered after RGNEF depletion (FIG. 21).

TABLE 4
LIST OF GENES DOWNREGULATED

| | Gene | log2fold | Fold | | |
|---|---|---|---|---|---|
| Ensembl | Symbol | change | Change | pvalue | padjust |
| ENSG00000214290 | C11orf93 | −2.83 | 0.14 | 6.02E−11 | 9.41E−09 |
| ENSG00000011201 | KAL1 | −2.62 | 0.16 | 2.42E−06 | 1.14E−04 |
| ENSG00000139971 | C14orf37 | −2.62 | 0.16 | 1.83E−08 | 1.63E−06 |
| ENSG00000137033 | IL33 | −2.43 | 0.19 | 8.00E−10 | 1.00E−07 |
| ENSG00000176840 | MIR7-3HG | −2.36 | 0.19 | 7.47E−05 | 2.02E−03 |
| ENSG00000074410 | CA12 | −2.3 | 0.2 | 1.49E−28 | 3.90E−25 |
| ENSG00000196639 | HRH1 | −2.29 | 0.2 | 3.10E−06 | 1.40E−04 |
| ENSG00000245651 | RP11-620J15.2 | −2.16 | 0.22 | 1.27E−07 | 8.73E−06 |
| ENSG00000070915 | SLC12A3 | −2.13 | 0.23 | 2.84E−05 | 9.38E−04 |
| ENSG00000188993 | LRRC66 | −2.08 | 0.24 | 8.70E−05 | 2.28E−03 |
| ENSG00000151322 | NPAS3 | −2.03 | 0.24 | 7.95E−07 | 4.36E−05 |
| ENSG00000082929 | C4orf6 | −1.98 | 0.25 | 3.04E−05 | 9.95E−04 |
| ENSG00000168505 | GBX2 | −1.97 | 0.25 | 1.35E−05 | 5.01E−04 |
| ENSG00000271367 | RP3-483K16.4 | −1.97 | 0.26 | 3.42E−04 | 6.87E−03 |
| ENSG00000162496 | DHRS3 | −1.95 | 0.26 | 3.38E−08 | 2.83E−06 |
| ENSG00000269916 | RP11-193M21.1 | −1.76 | 0.29 | 1.96E−03 | 2.66E−02 |
| ENSG00000126778 | SIX1 | −1.75 | 0.3 | 3.36E−04 | 6.77E−03 |
| ENSG00000256043 | CTSO | −1.74 | 0.3 | 6.95E−22 | 7.49E−19 |
| ENSG00000116171 | SCP2 | −1.63 | 0.32 | 1.67E−38 | 3.06E−34 |
| ENSG00000132854 | KANK4 | −1.62 | 0.33 | 2.47E−03 | 3.13E−02 |
| ENSG00000140279 | DUOX2 | −1.6 | 0.33 | 6.26E−04 | 1.10E−02 |
| ENSG00000008282 | SYPL1 | −1.59 | 0.33 | 7.07E−34 | 6.47E−30 |
| ENSG00000230082 | PRRT3-AS1 | −1.57 | 0.34 | 1.03E−03 | 1.60E−02 |
| ENSG00000265096 | AC073624.1 | −1.57 | 0.34 | 3.66E−22 | 4.46E−19 |
| ENSG00000174332 | GLIS1 | −1.55 | 0.34 | 6.14E−04 | 1.09E−02 |
| ENSG00000168539 | CHRM1 | −1.55 | 0.34 | 1.06E−05 | 4.12E−04 |
| ENSG00000176769 | TCERG1L | −1.54 | 0.34 | 3.18E−04 | 6.50E−03 |
| ENSG00000183615 | FAM167B | −1.52 | 0.35 | 7.27E−07 | 4.08E−05 |
| ENSG00000107623 | GDF10 | −1.5 | 0.35 | 7.54E−04 | 1.27E−02 |
| ENSG00000203401 | AC009061.1 | −1.5 | 0.35 | 1.80E−03 | 2.49E−02 |
| ENSG00000104419 | NDRG1 | −1.48 | 0.36 | 8.50E−30 | 2.59E−26 |
| ENSG00000256628 | RP11-454H13.5 | −1.46 | 0.36 | 4.31E−03 | 4.69E−02 |
| ENSG00000129625 | REEP5 | −1.46 | 0.36 | 6.79E−33 | 3.11E−29 |
| ENSG00000040608 | RTN4R | −1.46 | 0.36 | 3.39E−27 | 6.20E−24 |
| ENSG00000158270 | COLEC12 | −1.45 | 0.37 | 1.35E−08 | 1.26E−06 |
| ENSG00000253986 | CTC-756D1.3 | −1.45 | 0.37 | 1.87E−03 | 2.56E−02 |
| ENSG00000224008 | RP5-1010E17.2 | −1.44 | 0.37 | 1.16E−03 | 1.77E−02 |
| ENSG00000066382 | MPPED2 | −1.41 | 0.38 | 3.18E−03 | 3.78E−02 |
| ENSG00000137642 | SORL1 | −1.41 | 0.38 | 8.40E−21 | 8.09E−18 |
| ENSG00000242629 | USP6NL-IT1 | −1.4 | 0.38 | 1.99E−11 | 3.83E−09 |
| ENSG00000204174 | NPY4R | −1.39 | 0.38 | 1.01E−03 | 1.59E−02 |
| ENSG00000101265 | RASSF2 | −1.39 | 0.38 | 1.45E−19 | 1.15E−16 |
| ENSG00000111667 | USP5 | −1.39 | 0.38 | 4.11E−33 | 2.51E−29 |
| ENSG00000179388 | EGR3 | −1.38 | 0.38 | 1.98E−07 | 1.31E−05 |
| ENSG00000066248 | NGEF | −1.37 | 0.39 | 1.63E−04 | 3.76E−03 |
| ENSG00000270019 | RP11-141B14.1 | −1.36 | 0.39 | 1.19E−04 | 2.95E−03 |
| ENSG00000125249 | RAP2A | −1.35 | 0.39 | 4.23E−30 | 1.55E−26 |
| ENSG00000185585 | OLFML2A | −1.35 | 0.39 | 1.18E−15 | 4.96E−13 |
| ENSG00000053108 | FSTL4 | −1.35 | 0.39 | 2.11E−10 | 3.00E−08 |
| ENSG00000230606 | AC159540.1 | −1.32 | 0.4 | 8.25E−05 | 2.19E−03 |
| ENSG00000215475 | SIAH3 | −1.32 | 0.4 | 1.55E−04 | 3.60E−03 |
| ENSG00000115155 | OTOF | −1.32 | 0.4 | 8.89E−06 | 3.50E−04 |
| ENSG00000049283 | EPN3 | −1.32 | 0.4 | 4.73E−06 | 2.03E−04 |
| ENSG00000123096 | SSPN | −1.3 | 0.4 | 6.22E−16 | 2.84E−13 |
| ENSG00000117500 | TMED5 | −1.3 | 0.41 | 6.68E−28 | 1.36E−24 |
| ENSG00000013619 | MAMLD1 | −1.3 | 0.41 | 1.28E−11 | 2.57E−09 |
| ENSG00000237624 | OXCT2P1 | −1.28 | 0.41 | 1.48E−03 | 2.14E−02 |
| ENSG00000116183 | PAPPA2 | −1.27 | 0.42 | 8.99E−05 | 2.34E−03 |
| ENSG00000231691 | RP11-203F10.5 | −1.26 | 0.42 | 1.14E−03 | 1.75E−02 |
| ENSG00000229191 | RP11-168016.1 | −1.26 | 0.42 | 8.25E−04 | 1.36E−02 |
| ENSG00000135914 | HTR2B | −1.26 | 0.42 | 8.52E−09 | 8.36E−07 |
| ENSG00000125355 | TMEM255A | −1.26 | 0.42 | 4.01E−03 | 4.47E−02 |
| ENSG00000185168 | LINC00482 | −1.25 | 0.42 | 3.75E−04 | 7.40E−03 |
| ENSG00000163293 | NIPAL1 | −1.25 | 0.42 | 1.46E−04 | 3.43E−03 |
| ENSG00000179148 | ALOXE3 | −1.24 | 0.42 | 3.01E−06 | 1.37E−04 |
| ENSG00000181649 | PHLDA2 | −1.24 | 0.42 | 1.70E−04 | 3.86E−03 |
| ENSG00000102098 | SCML2 | −1.24 | 0.42 | 1.76E−18 | 1.15E−15 |
| ENSG00000160963 | COL26A1 | −1.23 | 0.43 | 3.95E−11 | 6.87E−09 |
| ENSG00000124215 | CDH26 | −1.21 | 0.43 | 5.07E−04 | 9.29E−03 |
| ENSG00000128242 | GAL3ST1 | −1.2 | 0.43 | 1.59E−11 | 3.13E−09 |
| ENSG00000142530 | FAM71E1 | −1.19 | 0.44 | 1.29E−04 | 3.15E−03 |
| ENSG00000166128 | RAB8B | −1.19 | 0.44 | 5.28E−28 | 1.21E−24 |
| ENSG00000078081 | LAMP3 | −1.19 | 0.44 | 1.04E−04 | 2.67E−03 |
| ENSG00000151287 | TEX30 | −1.19 | 0.44 | 6.09E−20 | 5.30E−17 |
| ENSG00000035141 | FAM136A | −1.19 | 0.44 | 4.87E−20 | 4.45E−17 |
| ENSG00000250999 | RP11-1379J22.5 | −1.18 | 0.44 | 1.15E−03 | 1.76E−02 |
| ENSG00000255433 | AP000479.1 | −1.17 | 0.44 | 1.12E−04 | 2.81E−03 |
| ENSG00000091536 | MYO15A | −1.17 | 0.44 | 1.23E−03 | 1.87E−02 |
| ENSG00000149403 | GRIK4 | −1.16 | 0.45 | 9.31E−05 | 2.42E−03 |
| ENSG00000005893 | LAMP2 | −1.16 | 0.45 | 3.40E−24 | 5.19E−21 |
| ENSG00000242052 | RP11-190C22.1 | −1.15 | 0.45 | 3.65E−03 | 4.17E−02 |
| ENSG00000232021 | LEF1-AS1 | −1.15 | 0.45 | 1.05E−06 | 5.59E−05 |
| ENSG00000169499 | PLEKHA2 | −1.15 | 0.45 | 1.84E−21 | 1.87E−18 |
| ENSG00000143196 | DPT | −1.15 | 0.45 | 6.87E−06 | 2.81E−04 |
| ENSG00000259556 | RP11-56B16.2 | −1.14 | 0.45 | 3.99E−04 | 7.75E−03 |
| ENSG00000074219 | TEAD2 | −1.13 | 0.46 | 4.76E−17 | 2.72E−14 |
| ENSG00000168792 | ABHD15 | −1.13 | 0.46 | 2.33E−17 | 1.38E−14 |
| ENSG00000171792 | RHNO1 | −1.13 | 0.46 | 6.07E−23 | 7.93E−20 |
| ENSG00000262884 | CTD-3060P21.1 | −1.13 | 0.46 | 1.12E−04 | 2.81E−03 |
| ENSG00000136026 | CKAP4 | −1.13 | 0.46 | 2.36E−23 | 3.32E−20 |
| ENSG00000104164 | BLOC1S6 | −1.13 | 0.46 | 2.97E−24 | 4.95E−21 |
| ENSG00000175318 | GRAMD2 | −1.12 | 0.46 | 3.35E−03 | 3.90E−02 |
| ENSG00000160131 | VMA21 | −1.12 | 0.46 | 1.65E−19 | 1.25E−16 |
| ENSG00000176945 | MUC20 | −1.12 | 0.46 | 1.36E−04 | 3.28E−03 |
| ENSG00000094755 | GABRP | −1.12 | 0.46 | 9.04E−04 | 1.45E−02 |
| ENSG00000166923 | GREM1 | −1.12 | 0.46 | 5.13E−05 | 1.50E−03 |
| ENSG00000068078 | FGFR3 | −1.12 | 0.46 | 1.24E−16 | 6.69E−14 |
| ENSG00000171786 | NHLH1 | −1.11 | 0.46 | 1.61E−04 | 3.72E−03 |
| ENSG00000229539 | RP11-119B16.2 | −1.11 | 0.46 | 1.69E−03 | 2.37E−02 |
| ENSG00000112276 | BVES | −1.11 | 0.46 | 2.42E−19 | 1.77E−16 |

TABLE 5
LIST OF GENES UPREGULATED

| | Gene | log2fold | Fold | | |
|---|---|---|---|---|---|
| Ensembl | Symbol | change | Change | pvalue | padjust |
| ENSG00000248923 | MTND5P11 | 1.76553479 | 3.4 | 4.95E−04 | 9.09E−03 |
| ENSG00000255236 | CTD-2655K5.1 | 1.562936342 | 2.95 | 5.80E−05 | 1.64E−03 |
| ENSG00000197421 | GGT3P | 1.558731085 | 2.95 | 1.18E−03 | 1.80E−02 |
| ENSG00000177791 | MYOZ1 | 1.495957645 | 2.82 | 2.45E−03 | 3.11E−02 |
| ENSG00000169715 | MT1E | 1.466503816 | 2.76 | 1.24E−06 | 6.47E−05 |
| ENSG00000170627 | GTSF1 | 1.464668443 | 2.76 | 1.60E−03 | 2.28E−02 |
| ENSG00000146285 | SCML4 | 1.382145828 | 2.61 | 2.50E−03 | 3.17E−02 |
| ENSG00000271698 | TMEM249 | 1.352301902 | 2.55 | 4.44E−05 | 1.34E−03 |
| ENSG00000262152 | LINC00514 | 1.294743495 | 2.45 | 2.96E−03 | 3.60E−02 |
| ENSG00000163377 | FAM19A4 | 1.274794351 | 2.42 | 1.07E−03 | 1.66E−02 |
| ENSG00000228798 | AP000473.5 | 1.193537722 | 2.29 | 1.59E−04 | 3.69E−03 |
| ENSG00000074660 | SCARF1 | 1.189034033 | 2.28 | 2.29E−03 | 2.97E−02 |
| ENSG00000234617 | SNRK-AS1 | 1.116917976 | 2.17 | 4.58E−03 | 4.92E−02 |
| ENSG00000198417 | MT1F | 1.106915309 | 2.15 | 9.55E−09 | 9.15E−07 |
| ENSG00000215218 | UBE2QL1 | 1.06371171 | 2.09 | 2.08E−11 | 3.93E−09 |
| ENSG00000172318 | B3GALT1 | 1.062887909 | 2.09 | 2.06E−03 | 2.76E−02 |
| ENSG00000162670 | FAM5C | 1.050085411 | 2.07 | 3.07E−04 | 6.33E−03 |
| ENSG00000180875 | GREM2 | 1.023675872 | 2.03 | 1.66E−15 | 6.59E−13 |
| ENSG00000145949 | MYLK4 | 1.013939329 | 2.02 | 3.52E−05 | 1.12E−03 |
| ENSG00000171759 | PAH | 0.984346868 | 1.98 | 1.97E−05 | 6.83E−04 |
| ENSG00000229178 | AC069513.4 | 0.972519469 | 1.96 | 3.86E−03 | 4.34E−02 |
| ENSG00000169026 | MFSD7 | 0.96276725 | 1.95 | 9.33E−05 | 2.42E−03 |
| ENSG00000158296 | SLC13A3 | 0.958972918 | 1.94 | 2.44E−03 | 3.10E−02 |
| ENSG00000260585 | RP13-192B19.2 | 0.956931454 | 1.94 | 7.65E−04 | 1.29E−02 |
| ENSG00000181392 | SYNE4 | 0.945323087 | 1.93 | 1.79E−03 | 2.48E−02 |
| ENSG00000214842 | RAD51AP2 | 0.866998076 | 1.82 | 4.35E−03 | 4.72E−02 |
| ENSG00000225857 | RP11-46A10.2 | 0.862763719 | 1.82 | 2.21E−06 | 1.06E−04 |
| ENSG00000173698 | GPR64 | 0.845176247 | 1.8 | 2.30E−11 | 4.13E−09 |
| ENSG00000169962 | TAS1R3 | 0.84217137 | 1.79 | 2.32E−03 | 2.99E−02 |
| ENSG00000184564 | SLITRK6 | 0.823385923 | 1.77 | 1.69E−05 | 6.02E−04 |
| ENSG00000184809 | C21orf88 | 0.821907863 | 1.77 | 4.49E−03 | 4.84E−02 |
| ENSG00000111199 | TRPV4 | 0.813988132 | 1.76 | 6.00E−05 | 1.69E−03 |
| ENSG00000050628 | PTGER3 | 0.795347303 | 1.74 | 1.46E−03 | 2.13E−02 |
| ENSG00000260645 | RP11-250B2.5 | 0.789121719 | 1.73 | 6.77E−04 | 1.17E−02 |
| ENSG00000198569 | SLC34A3 | 0.78257392 | 1.72 | 6.90E−07 | 3.92E−05 |
| ENSG00000145861 | C1QTNF2 | 0.767884359 | 1.7 | 1.38E−04 | 3.31E−03 |
| ENSG00000154127 | UBASH3B | 0.765366182 | 1.7 | 9.16E−04 | 1.46E−02 |
| ENSG00000181418 | DDN | 0.7525892 | 1.68 | 5.55E−07 | 3.26E−05 |
| ENSG00000238286 | SLC35E1P1 | 0.747648863 | 1.68 | 4.51E−03 | 4.86E−02 |
| ENSG00000069535 | MAOB | 0.742812516 | 1.67 | 6.10E−05 | 1.71E−03 |
| ENSG00000256304 | RP11-512M8.3 | 0.732826027 | 1.66 | 2.24E−03 | 2.92E−02 |
| ENSG00000063438 | AHRR | 0.701695611 | 1.63 | 7.85E−05 | 2.11E−03 |
| ENSG00000259555 | RP11-335K5.2 | 0.662657683 | 1.58 | 5.60E−04 | 1.01E−02 |
| ENSG00000146469 | VIP | 0.659565265 | 1.58 | 3.32E−04 | 6.74E−03 |
| ENSG00000159588 | CCDC17 | 0.655554622 | 1.58 | 4.43E−03 | 4.79E−02 |
| ENSG00000099250 | NRP1 | 0.655254558 | 1.57 | 2.48E−05 | 8.33E−04 |
| ENSG00000139508 | SLC46A3 | 0.63977988 | 1.56 | 9.35E−04 | 1.49E−02 |
| ENSG00000270441 | RP11-694115.7 | 0.627829265 | 1.55 | 1.08E−03 | 1.67E−02 |
| ENSG00000214595 | EML6 | 0.626285559 | 1.54 | 3.25E−08 | 2.76E−06 |
| ENSG00000233198 | RNF224 | 0.621375586 | 1.54 | 3.05E−04 | 6.32E−03 |
| ENSG00000118298 | CA14 | 0.620426165 | 1.54 | 2.02E−04 | 4.46E−03 |
| ENSG00000128617 | OPN1SW | 0.615684514 | 1.53 | 7.82E−06 | 3.17E−04 |
| ENSG00000269589 | AC006128.2 | 0.615066588 | 1.53 | 2.77E−03 | 3.42E−02 |
| ENSG00000143375 | CGN | 0.613311926 | 1.53 | 7.58E−06 | 3.09E−04 |
| ENSG00000145335 | SNCA | 0.610917487 | 1.53 | 4.46E−05 | 1.34E−03 |
| ENSG00000146674 | IGFBP3 | 0.604669666 | 1.52 | 1.68E−04 | 3.86E−03 |
| ENSG00000108924 | HLF | 0.594743633 | 1.51 | 9.97E−04 | 1.57E−02 |
| ENSG00000197191 | C9orf169 | 0.583655232 | 1.5 | 1.15E−03 | 1.77E−02 |
| ENSG00000181449 | SOX2 | 0.580810965 | 1.5 | 4.48E−04 | 8.48E−03 |
| ENSG00000173726 | TOMM20 | 0.580003705 | 1.49 | 1.78E−07 | 1.19E−05 |
| ENSG00000188641 | DPYD | 0.572744227 | 1.49 | 3.66E−07 | 2.24E−05 |
| ENSG00000183779 | ZNF703 | 0.572307314 | 1.49 | 1.24E−03 | 1.88E−02 |
| ENSG00000187240 | DYNC2H1 | 0.562710618 | 1.48 | 6.62E−05 | 1.83E−03 |
| ENSG00000176723 | ZNF843 | 0.559511996 | 1.47 | 3.35E−03 | 3.90E−02 |
| ENSG00000170577 | SIX2 | 0.556122917 | 1.47 | 1.01E−03 | 1.58E−02 |
| ENSG00000163794 | UCN | 0.545893053 | 1.46 | 3.72E−03 | 4.22E−02 |
| ENSG00000128849 | CGNL1 | 0.536803224 | 1.45 | 3.84E−04 | 7.55E−03 |
| ENSG00000182621 | PLCB1 | 0.533164664 | 1.45 | 8.07E−06 | 3.26E−04 |
| ENSG00000167994 | RAB3IL1 | 0.527997559 | 1.44 | 1.13E−04 | 2.82E−03 |
| ENSG00000163701 | IL17RE | 0.522678328 | 1.44 | 2.50E−03 | 3.17E−02 |
| ENSG00000066583 | ISOC1 | 0.52097441 | 1.43 | 8.90E−04 | 1.43E−02 |
| ENSG00000168348 | INSM2 | 0.51176753 | 1.43 | 7.22E−04 | 1.23E−02 |
| ENSG00000261578 | RP11-21L23.2 | 0.503941571 | 1.42 | 2.70E−04 | 5.74E−03 |
| ENSG00000173114 | LRRN3 | 0.495433962 | 1.41 | 1.93E−03 | 2.63E−02 |
| ENSG00000221866 | PLXNA4 | 0.494976985 | 1.41 | 6.21E−04 | 1.10E−02 |
| ENSG00000186301 | MST1P2 | 0.49030344 | 1.4 | 2.75E−03 | 3.41E−02 |
| ENSG00000128595 | CALU | 0.486184736 | 1.4 | 6.21E−06 | 2.58E−04 |
| ENSG00000154027 | AK5 | 0.484959499 | 1.4 | 9.26E−04 | 1.47E−02 |
| ENSG00000095739 | BAMBI | 0.482263484 | 1.4 | 8.64E−05 | 2.27E−03 |
| ENSG00000126368 | NR1D1 | 0.479121205 | 1.39 | 2.16E−04 | 4.71E−03 |
| ENSG00000136573 | BLK | 0.467986458 | 1.38 | 4.42E−04 | 8.41E−03 |
| ENSG00000138434 | SSFA2 | 0.46300658 | 1.38 | 2.11E−05 | 7.28E−04 |
| ENSG00000101888 | NXT2 | 0.462587135 | 1.38 | 3.17E−04 | 6.47E−03 |
| ENSG00000178105 | DDX10 | 0.457255566 | 1.37 | 7.51E−05 | 2.03E−03 |
| ENSG00000132970 | WASF3 | 0.45707779 | 1.37 | 6.41E−05 | 1.79E−03 |
| ENSG00000162004 | CCDC78 | 0.442023581 | 1.36 | 4.61E−03 | 4.94E−02 |
| ENSG00000106868 | SUSD1 | 0.441832572 | 1.36 | 4.74E−04 | 8.81E−03 |
| ENSG00000101445 | PPP1R16B | 0.441071115 | 1.36 | 1.02E−03 | 1.60E−02 |
| ENSG00000254064 | CTD-2530N21.4 | 0.435184161 | 1.35 | 4.16E−03 | 4.57E−02 |
| ENSG00000145919 | BOD1 | 0.432685446 | 1.35 | 3.36E−04 | 6.77E−03 |
| ENSG00000113580 | NR3C1 | 0.425062485 | 1.34 | 1.63E−03 | 2.31E−02 |
| ENSG00000179242 | CDH4 | 0.424036119 | 1.34 | 2.77E−03 | 3.42E−02 |
| ENSG00000088305 | DNMT3B | 0.422317618 | 1.34 | 4.84E−04 | 8.96E−03 |
| ENSG00000172382 | PRSS27 | 0.418477841 | 1.34 | 2.85E−03 | 3.50E−02 |
| ENSG00000121310 | ECHDC2 | 0.414251232 | 1.33 | 1.02E−03 | 1.60E−02 |
| ENSG00000163818 | LZTFL1 | 0.409030778 | 1.33 | 2.77E−03 | 3.42E−02 |
| ENSG00000113657 | DPYSL3 | 0.405039883 | 1.32 | 4.46E−04 | 8.46E−03 |
| ENSG00000164118 | CEP44 | 0.404039014 | 1.32 | 2.29E−04 | 4.95E−03 |
| ENSG00000150867 | PIP4K2A | 0.390964218 | 1.31 | 5.26E−04 | 9.54E−03 |
| ENSG00000162946 | DISC1 | 0.390637827 | 1.31 | 1.68E−03 | 2.35E−02 |

negative limitation removing any subject matter from the genus, regardless of whether or not the excised material is specifically recited herein.

In addition, where features or aspects of the disclosure are described in terms of Markush groups, those skilled in the art will recognize that the disclosure is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

1-23. (canceled)

24. A method for treating a condition associated with TDP-43 toxicity in a subject, the method comprising administering to the subject an effective amount of a TDP-43 antagonist, wherein the TDP-43 antagonist is one or more of (a) a peptide that binds and inhibits TDP-43, the peptide comprising an N-terminus of a Rho guanine nucleotide exchange factor (RGNEF), or (b) a substance or molecule that increases the endogenous expression of RGNEF in the subject, thereby treating the condition associated with TDP-43 toxicity.

25. The method of claim 24, wherein the TD-43 antagonist is (a), and the peptide has at least 70% homology to SEQ ID NO: 13.

26. The method of claim 24, wherein the TD-43 antagonist is (a), and the peptide contains SEQ ID NO:13.

27. The method of claim 26, wherein the peptide is about 1731 amino acids long.

28. The method of claim 24, wherein the peptide is a full length RGNEF.

29. The method of claim 24, wherein the TDP-43 antagonist is (a), and the peptide has at least 70% homology to SEQ ID NO: 16.

30. The method of claim 29, wherein the peptide comprising the N-terminus domain of the RGNEF is about 242 amino acids long.

31. The method of claim 24, wherein the peptide also comprises a Pleckstrin Homology (PH) domain of the RGNEF.

32. The method of claim 24, wherein the TDP-43 antagonist is (b), and the substance or molecule is a nucleic acid molecule that encodes a peptide comprising the N-terminus domain of the RGNEF.

33. The method of claim 32, wherein the nucleic acid molecule contains SEQ ID NO: 15.

34. The method of claim 24, wherein the TDP-43 antagonist is (b), and the substance or molecule is an antisense oligonucleotide that is complementary to a nucleic acid sequence that inhibits endogenous expression of RGNEF.

35. The method of claim 24, wherein the TDP-43 antagonist is (b), and the substance or molecule is a siRNA that degrades encoding mRNA that inhibits endogenous expression of RGNEF.

36. The method of claim 24, wherein the TDP-43 antagonist is (b), and the substance or molecule is a ribozyme that catalyzes cleavage of mRNA that inhibits expression of RGNEF in a cell.

37. The method of claim 24, wherein the condition associated with TDP-43 toxicity is Amyotrophic Lateral Sclerosis (ALS), and the method is a method of treating ALS in the subject.

38. The method of claim 24, wherein the condition associated with TDP-43 toxicity is neuron degeneration, and the method is a method of treating neuron degeneration in the subject.

39. The method of claim 24, wherein the condition is life expectancy, and the method is a method to increase life expectancy of the subject.

40. The method of claim 24, wherein the method further administering the TDP-43 antagonist in combination with an agent that enhances the effect of the TDP-43 antagonist.


SEQUENCE LISTING (16 sequences)

SEQ ID NO: 1 - DNA, 30 bp, Artificial Sequence
  Synthetic RGNEF-deltaGEF Forward primer
attggtaccatggagttgagctgcagcgaa

SEQ ID NO: 2 - DNA, 32 bp, Artificial Sequence
  Synthetic RGNEF-deltaGEF Reverse primer
taaggaggtgtttgatgacatccttctcctgc

SEQ ID NO: 3 - DNA, 34 bp, Artificial Sequence
  Synthetic RGNEF-deltaGEF Forward primer
tgtcatcaaacacctccttattaaacctgaccca

SEQ ID NO: 4 - DNA, 32 bp, Artificial Sequence
  Synthetic RGNEF-deltaGEF Reverse primer
agactcgagcaccttgaggtctacttgatgtt

SEQ ID NO: 5 - DNA, 30 bp, Artificial Sequence; identical to SEQ ID NO: 1
  Synthetic RGNEF-deltaRBD Forward primer

SEQ ID NO: 6 - DNA, 30 bp, Artificial Sequence
  Synthetic RGNEF-deltaRBD Reverse primer
caactggaggggctctagatggacgtcctc

SEQ ID NO: 7 - DNA, 30 bp, Artificial Sequence; identical to SEQ ID NO: 1
  Synthetic RGNEF-NH2 Forward primer

SEQ ID NO: 8 - DNA, 38 bp, Artificial Sequence
  Synthetic RGNEF-NH2 Reverse primer
tttctcgagtttgatgacatccttctcctgcctattac

SEQ ID NO: 9 - DNA, 36 bp, Artificial Sequence
  Synthetic RGNEF-COOH Forward primer
ataggtaccatgagacaggatgtcatttttgagcta

SEQ ID NO: 10 - DNA, 32 bp, Artificial Sequence; identical to SEQ ID NO: 4
  Synthetic RGNEF-COOH Reverse primer

SEQ ID NO: 11 - DNA, 32 bp, Artificial Sequence
  Synthetic RGNEF-NH2-deltaL-rich Forward primer
ataggtaccatgattcactcatcggaaacgct

SEQ ID NO: 12 - DNA, 38 bp, Artificial Sequence; identical to SEQ ID NO: 8
  Synthetic RGNEF-NH2-deltaL-rich Reverse primer

SEQ ID NO: 13 - protein, 1731 aa, Homo sapiens [sequence omitted]

SEQ ID NO: 14 - protein, 119 aa, Homo sapiens
  misc_feature (1)..(119): PH Domain
EERTLLYDGLVYWKTATGRFKDILALLLTDVLLFLQEKDQKYIFAAVDQKPSVISLQKLIAREVANEERGMFLISASSAGPEMYEIHTNSKEERNNWMRRIQQAVESCPEEKGGRTSES

SEQ ID NO: 15 - DNA, 726 bp, Homo sapiens
  misc_feature (1)..(726): Region of RGNEF that contains the Leu-rich domain
atggagttgagctgcagcgaagcacctctttacgggcagatgatgatctatgcgaagtttgacaaaaatgtgtatcttcctgaagatgctgagttttactttacttatgacggatctcatcagcgacatgtcatgattgcagagcgcatcgaggataacgttctccagtccagcgtcccaggccatgggcttcaggagacggtgacggtatctgtgtgcctctgctcggaaggttactctccggtgaccatgggctctggctcagtgacctacgtggacaacatggcttgcaggctggctcgtctgctggtgacgcaggccaatcgcctcacagcctgcagccaccagaccctgctgaccccatttgccttgacggcaggagcactgcctgccttggatgaggagctcgtgctggctctgacccatctggaattgcctctagagtggactgtgttgggaagttcttcacttgaagtatcttctcacagagaatctcttctacacctggctatgagatggggcctggctaaactttcccagttcttcttgtgtctcccggggggagtccaggccttggctttacccaacgaagagggtgccacaccattagacttagctttacgtgaaggacactccaagctggtggaagacgtcacaaattttcagggcagatggtccccaagcttctcccgagtgcagctcagtgaagaagcctccttgcattac

SEQ ID NO: 16 - protein, 242 aa, Homo sapiens
  misc_feature (1)..(242): Region of RGNEF that contains the Leu-rich domain
MELSCSEAPLYGQMMIYAKFDKNVYLPEDAEFYFTYDGSHQRHVMIAERIEDNVLQSSVPGHGLQETVTVSVCLCSEGYSPVTMGSGSVTYVDNMACRLARLLVTQANRLTACSHQTLLTPFALTAGALPALDEELVLALTHLELPLEWTVLGSSSLEVSSHRESLLHLAMRWGLAKLSQFFLCLPGGVQALALPNEEGATPLDLALREGHSKLVEDVTNFQGRWSPSFSRVQLSEEASLHY